(19) United States
(12) Patent Application Publication
(10) Pub. No.: US 2022/0390436 A1
(43) Pub. Date: Dec. 8, 2022

(54) T-CELL RECEPTOR NEOANTIGEN INTERACTION ANALYSIS VIA MICROFLUIDICS

(71) Applicant: Scribe Biosciences, San Francisco, CA (US)

(72) Inventors: Maithreyan Srinivasan, San Francisco, CA (US); Russell Cole, San Francisco, CA (US); Nathan Schoepp, San Francisco, CA (US)

(21) Appl. No.: 17/771,561

(22) PCT Filed: Oct. 26, 2020
(86) PCT No.: PCT/US20/57333

§ 371 Date: Apr. 25, 2022

Related U.S. Application Data
(60) Provisional application No. 62/925,869, filed on Oct. 25, 2019.
(60) Provisional application No. 62/980,598, filed on Feb. 24, 2020.

(51) Int. Cl. G01N 33/50; C12N 15/10; B01L 3/00

(52) U.S. Cl. CPC G01N 33/5047; G01N 33/5011; C12N 15/1037; C12N 15/1065; B01L 3/502761; B01L 2300/0645; B01L 2400/0424; B01L 2200/0652; B01L 2200/16; B01L 2300/0864; B01L 2200/0673

(57) ABSTRACT

The present invention provides compositions, systems, kits, and methods for analyzing the interaction of T-cells and neoantigen presenting cells (and other cells) via discrete entity (e.g., droplet) microfluids. In certain embodiments, a microfluidic device is used to merge a discrete entity containing a T-cell, and a discrete entity containing a neoantigen presenting cell, at a merger region via a trapping element in order to generate a combined discrete entity. In particular embodiments, at least one thousand of such combined discrete entities are formed in about one second. In some embodiments, whether the receptor on the T-cell sufficiently binds the neoantigen to activate the T-Cell is detected (e.g., via detection of cytokine or granzyme B release). In certain embodiments, provided herein are methods for identifying polyfunctional T-cells or NK-cells, as well as methods of screening for such cells that would be cytotoxic if injected into a subject.

The present application claims priority to U.S. Provisional application Ser. No. 62/980,598 filed Feb. 24, 2020, and 62/925,869 filed Oct. 25, 2019; both of which are herein incorporated by reference in their entireties.

### FIELD OF THE INVENTION

The present invention provides compositions, systems, kits, and methods for analyzing the interaction of T-cells and neoantigen presenting cells (and other cells) via discrete entity (e.g., droplet) microfluids. In certain embodiments, a microfluidic device is used to merge a discrete entity containing a T-cell, and a discrete entity containing a neoantigen presenting cell, at a merger region via a trapping element in order to generate a combined discrete entity. In particular embodiments, at least one thousand of such combined discrete entities are formed in about one second. In some embodiments, whether the receptor on the T-cell sufficiently binds the neoantigen to activate the T-Cell is detected (e.g., via detection of cytokine release or granzyme B). In certain embodiments, provided herein are methods for identifying polyfunctional T-cells or NK-cells, as well as methods of screening for such cells that would be cytotoxic if injected into a subject.

### BACKGROUND

Cancer has become the leading cause of death before the age of 70 in 48 out of 172 countries [1]. Earliest documented evidence of cancer dates back over 3000 years when treatment options were limited only to palliative care [2]. Our continued search for the causes of cancer has identified several contributory factors—these include environmental factors, such as exposure to tobacco smoke correlating with increased incidence of lung cancer, and inherited mutations, such as the higher incidence of breast/ovarian cancer with BRCA1 and BRCA2 mutations. In addition, recent studies by Tomasetti et. al. [3] suggest that random mutations caused by replication errors contribute to over two-thirds of observed gene mutations in human cancers. Environmental/hereditary factors and replication errors ultimately result in changes either in the protein coding regions that alter/abolish protein function or in non-coding regions that result in altered levels of expression. Abnormal expression of proteins in cancers frequently involve the pathological overexpression of certain proteins in cancers that are typically expressed at low levels in normal tissues. Many of these proteins, called tumor associated antigens (TAAs), are targets for T-cells isolated from tumor infiltrating lymphocytes (TILs), draining lymph nodes and peripheral blood. However, because normal cells also express these TAAs, central and peripheral tolerance mechanisms can to lead to low affinity T-cell receptors (TCRs) on responsive T-cells. Avoiding these mechanisms by engineering high-affinity TCRs for these TAAs has successfully been shown to shrink tumors but also leads to severe side-effects [4][5]. Therefore, reliably identifying and characterizing antigens that are specifically expressed in tumors and not in normal cells can potentially generate durable immune responses that exhibit minimal tolerance and side effects.

Such antigens, referred to as neoantigens, arise from altered amino-acid sequences in proteins generated by various mechanisms [6]. Each cancer patient has a unique set of neoantigens which necessitates a level of personalization to any cell-based treatment. This requires characterizing the specific neoantigens, identifying the cognate T-cells to these neoantigens, manufacturing the identified T-cells, and administering these cells back into the patient. Such personalized therapies, while effective, add up to $1.5 million in therapy and hospitalization costs [7]. Thus, there is a critical need to decrease costs of personalized therapies and eventually transition to an “off-the-shelf” T-cell drug cocktail for patients who present a list of matched immunogenic neoantigens. However, the biggest bottleneck to decreasing cell therapy costs is the lack of high throughput single-cell technologies to evaluate the hundreds of patient-specific mutations for their ability to act as neoantigens and determine the cognate T-cell receptors (TCRs) through functional assays.

### SUMMARY OF THE INVENTION

The present invention provides compositions, systems, kits, and methods for analyzing the interaction of T-cells and neoantigen presenting cells via discrete entity (e.g., droplet) microfluids. In certain embodiments, a microfluidic device is used to merge a discrete entity containing a T-cell, and a discrete entity containing a neoantigen presenting cell, at a merger region via a trapping element in order to generate a combined discrete entity. In particular embodiments, at least one thousand of such combined discrete entities are formed in about one second. In some embodiments, whether the receptor on the T-cell sufficiently binds the neoantigen to activate the T-Cell is detected (e.g., via detection of cytokine, granzyme B, or CD107a release). In certain embodiments, wherein the combined discrete entity further comprises detection reagents for detecting activation molecule release from said T-cell when it is activated, wherein said activation molecule is selected from: a cytokine, CD107a, and Granzyme B. In certain embodiments, provided herein are methods for identifying polyfunctional T-cells or NK-cells, as well as methods of screening for such cells that would be cytotoxic if injected into a subject.

In some embodiments, provided herein are methods comprising: a) flowing a first or second discrete entity in a carrier fluid in a microfluidic device, wherein the microfluidic device comprises: i) an inlet channel, ii) a sorting channel in fluid communication with the inlet channel, iii) first and second outlet channels in fluid communication with the sorting channel, wherein the first outlet channel comprises a merger region, iv) a sorting element positioned in proximity to the sorting channel, and v) a trapping element positioned in proximity to the merger region, and wherein the first discrete entity comprises at least one surface display cell (SD cell) and is free of other types of cells, wherein each SD cell comprises: i) an outer surface displaying a polypeptide, wherein the polypeptide comprises at least one neoantigen, and ii) a first detectable label; and wherein the second discrete entity comprises at least one T-cell and is free of other types of cells, wherein the T-cell comprises: i) a T-cell receptor (TCR), ii) nucleic acid encoding the TCR, and iii) a second detectable label; and wherein the flowing causes the first or second discrete entity to pass through the inlet channel to the sorting channel; b) selectively sorting the first or second discrete entity in the sorting channel to the first outlet channel; c) trapping the first or second discrete entity in the merger region via the trapping element; and d) repeating steps a)-c) such that both the first and second discrete entities are trapped at the merger region and combine to form a first combined discrete entity, wherein the repeating steps a)-c) overlaps in time, or is after, when the steps a)-c) are performed.

In some embodiments, provided herein are sorting methods comprising: a) flowing a plurality of aqueous discrete entities in an oil carrier fluid in an emulsion inlet channel of a microfluidic device, wherein said inlet channel feeds into an emulsion-aqueous junction, wherein: i) an aqueous inlet channel feeds into said emulsion-aqueous junction; ii) an aqueous outlet channel branches off of said emulsion-aqueous junction; and iii) an emulsion outlet channel branches off of said emulsion-aqueous junction, wherein an aqueous carrier fluid flows from said aqueous inlet channel to said emulsion-aqueous junction to said aqueous outlet channel and a first electrical signal is applied to said aqueous carrier fluid, wherein each of said plurality of aqueous discrete entities in said oil carrier merge into said aqueous carrier fluid at said emulsion-aqueous junction and flow out said aqueous outlet channel when said first electrical signal is applied to said aqueous carrier fluid, b) detecting at least one of said discrete entities as desired (desired discrete entity) by detecting at least one agent present in said desired discrete entity prior to it reaching said emulsion-aqueous junction, and c) changing said first electrical signal to a second electrical signal applied to said aqueous carrier fluid such that said desired discrete entity is prevented from merging into said aqueous carrier fluid at said emulsion-aqueous junction, and instead, flows in said carrier oil into said emulsion outlet. In certain embodiments, the methods further comprise: d) processing said desired discrete entity as described herein (e.g., in SD cell-T-cell merging methods, or spheroid assembly, or sequencing methodologies, etc.). Descriptions of emulsion-aqueous junctions, and changing electrical charge of the aqueous stream, are found in U.S. Pat. No. 8,828,210; Romero et al., PNAS, 2015, 112 (23): 7159-7164; and Fidalgo et al., Angew. Chem. Int. Ed. 2008, 47, 2042-2045; all of which are herein incorporated by reference in their entireties.

In certain embodiments, one and only one T-cell is present in the discrete entity. In particular embodiments, if multiple T-cells are present in the discrete entity, they are clonal (identical) cells. In other embodiments, one and only one SD cell is present in the discrete entity. In some embodiments, if multiple SD cells are present in the discrete entity, they are clonal (identical) cells.

In certain embodiments, the methods further comprise: e) detecting directly or indirectly, in the first combined discrete entity, whether the TCR on the T-cell sufficiently binds the neoantigen on the SD cell to activate the T-Cell (e.g., as detected by cytokine, granzyme B, or CD107a release by the T-cell). In some embodiments, the detecting is performed when the first combined discrete entity is at the merger region. In other embodiments, the methods further comprise: releasing the first combined discrete entity from the merger region such that it flows into a downstream area. In further embodiments, the detecting is performed when the first combined discrete entity is at the downstream area. In additional embodiments, the downstream area is a collection area or a receptacle external to said microfluidic device.

In further embodiments, the SD cell, prior to step a) has been pulsed with the neoantigen. In other embodiments, the identity of the neoantigen is known prior to performing the method. In certain embodiments, the SD cell further comprises: iii) a nucleic acid sequence encoding the neoantigen. In additional embodiments, the nucleic acid sequence is from a library of nucleic acid sequences encoding different neoantigens. In particular embodiments, the SD cell comprises an antigen presenting cell (APC) (e.g., macrophage, dendritic cells, and B cell). In further embodiments, the SD cell: comprises one or more nucleic acid sequences encoding an MHC sequence and the neoantigen (e.g., an HLA sequence and the neoantigen).

In some embodiments, the TCR of the T-cell is a chimeric antigen receptor. In other embodiments, the TCR of the T-cell is endogenous to said T-cell or a TRC synthesized as part of a library. In certain embodiments, the combined discrete entity further comprises detection reagents for detecting cytokine release from the T-cell when it is activated. In other embodiments, the detection reagents comprise first and second anti-cytokine antibodies, first and second anti-granzyme B antibodies, and/or first and second anti-CD107a antibodies. In further embodiments, the second antibody is detectably labeled and wherein the first antibodies are attached to a bead. In some embodiments, the detection reagents comprise first and second anti-activation molecule antibodies.

In other embodiments, steps a)-d) are performed: A) in 2 milliseconds (mS) or less; B) is about 1 mS; C) in about 0.5-1.0 mS. In further embodiments, the methods further comprise repeating steps a)-d) at least 99 times such that a total of at least 100 combined discrete entities are formed. In some embodiments, the methods further comprise repeating steps a)-d) at least 999 times such that a total of at least 1000 combined discrete entities are formed. In other embodiments, the methods further comprise repeating steps a)-d) at least 9999 times such that a total of at least 10000 combined discrete entities are formed. In other embodiments, the methods further comprise repeating steps a)-d) at least 99,999 times such that a total of at least 100,000 combined discrete entities are formed. In particular embodiments, the 100 or the 1000 discrete entities are formed: A) in 2 seconds or less; B) is about 1 second; C) in about 30-60 seconds. In other embodiments, the 10,000 or the 100,000 discrete entities are formed: A) in 20 seconds or less; B) is about 10 seconds; C) in about 300-600 seconds.

In particular embodiments, the methods further comprise: merging a third discrete entity with the first combined entity to generate a first further combined entity, wherein the third discrete entity comprises a PD1 inhibitor, or the PD1 inhibitor is already present in the first or second discrete entity. In some embodiments, the methods further comprise: e) detecting directly or indirectly, in the first combined discrete entity, that the neoantigen binds the TCR thereby activating the T-Cell. In particular embodiments, the methods further comprise: merging a third discrete entity with the first combined entity to generate a first further combined entity, wherein the third discrete entity comprises a lysis buffer, and wherein the at least one SD cell and the at least one T-cell are lysed inside the first further combined entity. In additional embodiments, the nucleic acid encoding the TCR is at least partially sequenced and/or wherein a nucleic acid sequence encoding the neoantigen is present and is at least partially sequenced. In certain embodiments, the methods further comprise: merging a fourth discrete entity with the first further combined entity to generate a first additionally combined entity, wherein the fourth discrete entity comprises: barcoded oligonucleotides and a polymerase. In some embodiments, the barcoded oligonucleotide comprises barcoded template switch oligonucleotides (BTSOs), and wherein the polymerase comprises reverse transcriptase. In further embodiments, the BTSOs are linked to a solid support bead via a photocleavable linker. In other embodiments, the first additional combined entity further comprises primers specific for the alpha and/or beta regions of the TCR. In other embodiments, the first additional combined entity further comprises primers specific for a nucleic acid sequence encoding the neoantigen.

In certain embodiments, the first and/or second detectable marker comprises a fluorescent protein. In other embodiments, the at least one SD cell and/or the at least one T-cell are mammalian cells or human cells. In some embodiments, the methods further comprise releasing the first combined discrete entity from the discrete entity merger region by deactivating, decreasing, or reversing the trapping element such that first combined discrete entity flows out of the first outlet channel. In particular embodiments, the first discrete entity is flowed through a first inlet channel and the second discrete entity is flowed through a second inlet channel.

In other embodiments, the sorting element comprises a first sorting electrode that exert an electromagnetic force sufficient to sort a discrete entity in the sorting channel to the first outlet channel. In some embodiments, the electromagnetic force is a dielectrophoretic force. In additional embodiments, the electromagnetic force is an electrophoretic force. In additional embodiments, the microfluid device further comprises a second and/or third sorting electrode. In further embodiments, the first and second sorting electrodes are configured such that the first and second sorting electrodes form a bipolar electrode pair and the first trapping electrode is positively charged. In certain embodiments, the first and second sorting electrodes are positioned on opposite sides of the sorting channel. In additional embodiments, at least one of the following applies: i) the first sorting electrode is positioned closer to the sorting channel than the second sorting electrode, ii) the second sorting electrode is positioned closer to the sorting channel than the first sorting electrode, iii) the distance between an end of the first sorting electrode, the second sorting electrode, or both and an interior wall of the sorter channel is between approximately 1 μm and approximately 100 μm, iv) the distance between the first sorting electrode and the second sorting electrode is approximately 25 μm to approximately 500 μm, v) the first sorting electrode and the second sorting electrode are connected to an alternating current electrical source with a frequency of approximately 0.1 kHz to approximately 100 kHz and a voltage of approximately 10 V to approximately 10,000 V, vi) each sorting electrode comprises a liquid electrode, vii) each sorting liquid electrode comprise one or more liquid channels imbedded in the method and filled with conductive media, and/or viii) the sorting element comprises a valve, a surface wave sorting element, an acoustic streaming element, or a combination thereof.

In certain embodiments, the trapping element exerts an electromagnetic force, exerts a mechanical force, or a combination thereof sufficient to trap the first and second discrete entities in the discrete entity merger region for a time sufficient for the discrete entities to combine to form a combined discrete entity. In other embodiments, the trapping element comprises a first trapping electrode that exerts an electromagnetic force sufficient to trap discrete entities in the merger region for a time sufficient for discrete entities to combine to form a combined discrete entity. In other embodiments, the electromagnetic force is a dielectrophoretic force. In further embodiments, the electromagnetic force is an electrophoretic force.

In particular embodiments, microfluidic device further comprises a second and/or third trapping electrode. In other embodiments, the first and second trapping electrodes are configured such that the first and second trapping electrodes form a bipolar electrode pair and the first trapping electrode is positively charged. In certain embodiments, at least one of the following applies: i) the first and second sorting electrodes are positioned on the same side of the sorting channel, ii) the first trapping electrode is positioned closer to the first outlet channel than the second trapping electrode, or the second trapping electrode is positioned closer to the first outlet channel than the first trapping electrode, iii) the distance between an end of the first trapping electrode, the second trapping electrode, or both and an interior wall the first outlet channel is between approximately 10 μm and approximately 50 μm, iv) the distance between the first trapping electrode and the second trapping electrode is approximately 25 μm to approximately 500 μm, v) the distance is approximately 50 μm to approximately 200 μm, vi) the first trapping electrode and the second trapping electrode are connected to an alternating current electrical source with a frequency of approximately 0.1 kHz to approximately 100 kHz and a voltage of approximately 10 V to approximately 10,000 V, vii) the frequency is approximately 1 kHz to approximately 50 kHz, viii) each trapping electrode comprises a liquid electrode, ix) each trapping liquid electrode comprise one or more liquid channels imbedded in the method and filled with conductive media, x) the first trapping electrode electrodes extends along the first outlet channel downstream of the discrete entity merger region, and/or xi) the second trapping electrode extends along the first outlet channel downstream of the discrete entity merger region.

In certain embodiments, the electrical trapping forces employed herein are modified. For example, in some embodiments, it is advantageous to modify the electrical signal sent to trapping electrodes as assembled droplets grow and affect the physics of droplet trapping and merging. In some embodiments, it is advantageous to increase the trapping voltage with each droplet added to overcome electrical shielding effects of the droplet. For instance, the signal applied to the electrical traps could increase from several hundred volts to several thousand volts during each droplet assembly. Similarly, in some cases it may be advantageous to reduce the voltage applied to an electrical trap to mitigate secondary field effects and because the need for a DEP force to move a droplet all the way to the channel wall is no longer needed. In certain embodiments, the voltage is reduced from an initial trapping force of several thousand volts to a minimal retention and merging voltage of several hundred volts as droplets are combined in the trap. Because electrokinetic forces can be frequency dependent, it may be appropriate to modify the frequency of the trapping signal as a means to achieve similar aims and thereby increase or reduce the applied electrical forces as droplets are added.

In particular embodiments, the sorting channel defines a concentric or approximately concentric flow path, and wherein a portion of the first sorting electrode is located at the center of the concentric or approximately concentric flow path. In some embodiments, the sorting element is positioned closer to the first outlet channel than to the second outlet channel. In other embodiments, the microfluidic device further comprises a partial height flow divider positioned in the sorting channel, wherein the partial height flow divider is configured to direct a discrete entity towards the first outlet channel or the second outlet channel. In certain embodiments, the height of the partial height flow divider is approximately 50% to 75% of the height of the sorting channel.

In some embodiments, the discrete entity merger region comprises a feature selected from the group consisting of: a geometric change in a dimension of the first outlet channel, a flow obstacle, a flow divider, a laminating fluid inlet, a valve, or a combination thereof. In other embodiments, the discrete entity merger region comprises a geometric change in a dimension of the first outlet channel, and wherein the geometric change comprises an increase in the cross-sectional area of the first outlet channel. In particular embodiments, the discrete entity merger region comprises a geometric change, and wherein the geometric change comprises a recess in a wall of the first outlet channel. In other embodiments, the discrete entity merger region comprises a laminating fluid inlet channel configured such that flowing laminating fluid through the laminating fluid inlet channel will direct a discrete entity in the discrete entity merger region towards a trapping electrode. In further embodiments, the inlet channel comprises an upstream region located between the sorting channel and the discrete entity merger region, and wherein the change in cross-sectional area is such that the discrete entity merger region has a larger cross-sectional area than the upstream region. In additional embodiments, the discrete entity merger region has a triangular shape, an approximately triangular shape, a trapezoidal shape, or an approximately trapezoidal shape defined by channel walls.

In some embodiments, the discrete entity merger region comprises a valve, wherein the valve is a membrane valve configured to impede the flow of a discrete entity past the discrete entity merger region while allowing flow of the carrier fluid past the discrete entity merger region in a first state, and wherein the membrane valve is configured to release the discrete entity or a combined discrete entity in a second state. In certain embodiments, the microfluidic device further comprises a spacer fluid channel in fluid communication with the inlet channel, wherein the spacer fluid channel is configured such that flowing spacer fluid through the spacer fluid channel causes spacer fluid to be located between the first and second discrete entities flowing through the inlet channel, thereby maintaining or increasing the distance between the first and second discrete entities, and thereby allowing each of first and second discrete entities to be independently sorted or not sorted.

In particular embodiments, the first and second discrete entities are droplets. In other embodiments, the droplets comprise an aqueous fluid which is immiscible in the carrier fluid. In additional embodiments, the carrier fluid comprises oil. In certain embodiments, the carrier fluid is an aqueous fluid and the droplets comprise a fluid which is immiscible with the carrier fluid. In additional embodiments, the discrete entities have a dimension of from about 1 μm to about 1000 μm (e.g., 1 . . . 50 . . . 300 . . . 1000). In other embodiments, the discrete entities have a diameter of from about 1 μm to 1000 μm (e.g., 1 . . . 50 . . . 300 . . . 1000). In further embodiments, the discrete entities have a volume of from about 1 femtoliter to about 1000 nanoliters (e.g., 1 . . . 50 . . . 300 . . . 1000), or from 10 to 800 picoliters.

In some embodiments, provided herein are compositions, systems, and kits comprising: a) first and second discrete entities in a carrier fluid, and/or b) a combined discrete entity in a carrier fluid, wherein the combined discrete entity is a combination of the first and second discrete entities, wherein the first discrete entity comprises at least one surface display cell (SD cell) and is free of other types of cells, wherein each SD cell comprises: i) an outer surface displaying a polypeptide, wherein the polypeptide comprises at least one neoantigen, and ii) a first detectable label; and wherein the second discrete entity comprises at least one T-cell and is free of other types of cells, wherein the T-cell comprises: i) a T-cell receptor (TCR), ii) nucleic acid encoding the TCR, and iii) a second detectable label.

In certain embodiments, the compositions, systems, and kits further comprise: c) a microfluidic device comprising: i) an inlet channel, ii) a sorting channel in fluid communication with the inlet channel, iii) first and second outlet channels in fluid communication with the sorting channel, wherein the first outlet channel comprises a merger region, iv) a sorting element positioned in proximity to the sorting channel, and v) a trapping element positioned in proximity to the merger region. In other embodiments, the compositions, systems, and kits further comprise a plurality of the first discrete entities and/or a plurality of the second discrete entities. In some embodiments, each of the first and second discrete entities comprises a droplet, and/or wherein the combined discrete entity comprises a droplet. In further embodiments, the droplet comprise an aqueous fluid in the carrier fluid. In other embodiments, the carrier fluid comprises oil.

In other embodiments, at least one of the first and second discrete entities, or the combined discrete entity, further comprises at least one of the following: a bead, primer, barcode sequence, template switching oligonucleotide (TSO), or a reverse transcriptase. In particular embodiments, the SD cell has been pulsed with the neoantigen. In additional embodiments, the SD cell further comprises: iii) a nucleic acid sequence encoding the neoantigen.

In further embodiments, the SD cell comprises an antigen presenting cell (APC). In further embodiments, the SD cell: comprises a nucleic acid sequence encoding an MHC sequence and the neoantigen. In further embodiments, the TCR of the T-cell is a chimeric antigen receptor. In further embodiments, the TCR of the T-cell is endogenous to the T-cell. In some embodiments, at least one of the first and second discrete entities, or the combined discrete entity, further comprises detection reagents for detecting cytokine, granzyme B, or CD107a release from the T-cell when it is activated. In other embodiments, the detection reagents comprise first and second anti-cytokine antibodies, first and second anti-granzyme B antibodies, and/or first and second anti-CD107a antibodies. In particular embodiments, the second antibody is detectably labeled and wherein the first antibodies are attached to a bead. In certain embodiments, one and only one T-cell is present in first or second discrete entity or the combined discrete entity. In further embodiments, one and only one SD cell is present in first or second discrete entity or the combined discrete entity.

In some embodiments, provided herein are methods comprising: a) flowing a plurality of discrete entities in a carrier fluid in a microfluidic device, wherein the microfluidic device comprises: i) a sorting channel, ii) first and second outlet channels in fluid communication with the sorting channel, iii) a collection area in fluid communication with the first outlet channel, and iv) a discard area in fluid communication with the second outlet channel, wherein each of the plurality of discrete entities comprises: i) an activated cell which is an activated T-cell or activated Natural Killer cell (NK cell), and ii) detection reagents for detecting interferon-gamma (IFN-γ) release and/or interleukin-6 (IL-6) release from the activated cell; b) detecting directly or indirectly, in each of the plurality of discrete entities when present in the sorting channel whether the activated cell: i) releases IFN-γ (IFN-γ positive discrete entity), or ii) does not release IL-6 (IL-6 negative discrete entity), or iii) releases IFN-γ and does not release IL-6 (IFN-γ positive and IL-6 negative discrete entity), c) selectively sorting each of the plurality of discrete entities in the sorting channel: i) to the first outlet channel and into the collection area if the discrete entity is an IFN-γ positive discrete entity, and to the second outlet channel and into the discard area if the discrete entity is not an IFN-γ positive discrete entity, thereby generating a population of IFN-γ positive discrete entities in the collection area; and/or ii) to the first channel and into the collection area if the discrete entity is an IL-6 negative discrete entity, and to the second channel and into the discard area if the discrete entity is not an IL-6 negative discrete entity, thereby generating a population of IL-6 negative discrete entities in the collection area; and/or iii) to the first channel and into the collection area if the discrete entity is an IFN-γ positive and IL-6 negative discrete entity, and to the second channel and into the discard area if the discrete entity is not IFN-γ positive and IL-6 negative, thereby generating a population of IFN-γ positive and IL-6 negative discrete entities in the collection area. In further embodiments, the methods further comprise: d) treating at least a portion of; i) the population of IFN-γ positive discrete entities, and/or ii) the population of IL-6 negative discrete entities, and/or iii) the population of IFN-γ positive and IL-6 negative discrete entities; under conditions such that such that some or all of the activated cells therein are each transduced with a vector encoding a chimeric antigen receptor thereby generating: i) a population of IFN-γ positive CAR cells (CAR T cells or CAR NK cells), and/or ii) a population of IL-6 negative CAR cells, and/or iii) a population of IFN-γ positive, IL-6 negative, CAR cells (CAR T cells or CAR NK cells).

In particular embodiments, provided herein are methods comprising: a) flowing a plurality of discrete entities in a carrier fluid in a microfluidic device, wherein the microfluidic device comprises: i) a sorting channel, ii) first and second outlet channels in fluid communication with the sorting channel, iii) a collection area in fluid communication with the first outlet channel, and iv) a discard area in fluid communication with the second outlet channel, wherein each of the plurality of discrete entities comprises: i) an activated cell which is an activated T-cell or activated NK cell, and ii) detection reagents for detecting release of at least two types of cytokines from the activated cell; b) detecting directly or indirectly, in each of the plurality of discrete entities when present in the sorting channel whether the activated cell releases the at least two types of cytokines (polyfunctional discrete entities) or does not release the at least two types of cytokines (non-polyfunctional discrete entities); and c) selectively sorting each of the plurality of discrete entities in the sorting channel: i) to the first outlet channel and into the collection area if the discrete entity is a polyfunctional discrete entity, and ii) to the second outlet channel and into the discard area if the discrete entity is a non-polyfunctional discrete entity, thereby generating a population of polyfunctional discrete entities in the collection area.

In certain embodiments, the activated cells are CAR T-cells or TCR T-cells or CAR NK cells. In some embodiments, each of the plurality of discrete entities contains only one of the activated cells (one of the activated T-cells or activated NK cells). In particular embodiments, each of the plurality of discrete entities is in the form of a droplet. In further embodiments, the droplet comprises an emulsion. In other embodiments, the treating comprises breaking the emulsion of each of plurality of discrete entities prior to the activated cells being transduced by the vector.

In some embodiments, each of the discrete entities comprises at least one activation molecule selected from the group consisting of; i) an anti-CD3 antibody, ii) an active fragment of the anti-CD3 antibody, iii) an anti-CD28 antibody, and iv) an active fragment of the anti-CD28 antibody. In other embodiments, the methods further comprise after c), but before d) flowing a fraction of; i) the population of IFN-γ positive discrete entities, and/or ii) the population of IL-6 negative discrete entities, and/or the IFN-γ positive and IL-6 negative discrete entities; in a carrier fluid in a microfluidic device such that reagent-containing discrete entities merge with the IFN-γ positive discrete entities and/or the IL-6 negative discrete entities and/or the IFN-γ positive and IL-6 negative discrete entities to generate a population of combined entities, wherein the reagent-containing discrete entities comprises lysis buffer and sequencing reagents. In some embodiments, the sequencing reagents comprise barcoded oligonucleotides and a polymerase. In additional embodiments, the barcoded oligonucleotide comprises barcoded template switch oligonucleotides (BTSOs), and wherein the polymerase comprises reverse transcriptase. In certain embodiments, the BTSOs are linked to a solid support bead via a photocleavable linker.

In other embodiments, the methods further comprise prior to step d) performing expression analysis sequencing on at least some of the activated cells from the population of combined entities. In some embodiments, the expression analysis generates data regarding at least cytokine gene toxic to a patient if over expressed by an activated cell when injected into a subject. In further embodiments, the at least one cytokine is LGALS1. In certain embodiments, the expression analysis generates data regarding an inflammatory signature profile that indicates the activated cell is toxic to a patient if injected into a subject. In some embodiments, the expression analysis generates data regarding expression levels for at least two beneficial cytokines, wherein over-expression of the at least two cytokines identifies an activated cell as polyfunctional. In further embodiments, the expression analysis generates data regarding a polyfunctional signature profile. In other embodiments, the polyfunctional signature profile comprises at least two genes selected from the group consisting of: ANXA1, CCL1, CCL3, CCL4, CCL5, CD40LG, CSF2, GZMA, GZMB, ICOS, IFNG, IL2, IL2RA, IL13, IL32, LCK, TNFRSF9, TNFRSF18, TNFRSF4, and TNFRSF14. In certain embodiments, the expression analysis generates data regarding activated cell identity for at least one, or all, of the following genes: CCND2, CD2, CD28, CD247, CD3D, CD3E, CD3G, CD44, CD7, CD96, TRAC, TRAV29DV5, TRBC1, TRBV12-3, TRBV20-1, TRBV5-1, and TRBV7-6.

### DESCRIPTION OF THE FIGURES

FIG. 1 provides a block schematic diagram of an example microfluidic device having an inlet channel, a sorting channel, a sorting element, first and second outlet channels, a trapping element, a discrete entity merger region, and upstream and a downstream regions.

FIG. 2 provides an image of a microfluidic device having a spacer fluid channel, a bias fluid channel, a laminating oil inlet channel, a concentric sorter channel, a flow divider, and a recess according to embodiments of the present disclosure.

FIG. 3 provides images of a microfluidic device having a concentric sorter channel, a recess, and an approximately triangular downstream region according to embodiments of the present disclosure.

FIG. 4, panels i-iv, show a zoomed-out view of an integrated droplet sorter-combiner. A droplet with a desired fluorescent signature is detected as it enters the droplet sorting region (i), the sorting electrode is actuated to redirect the drop towards the assembly lane (ii), and the sorted droplet merges with the droplet-in-assembly at the DEP trap (iii). Following assembly, the DEP trap is turned off to release the droplet (iv). Panels v-viii show a close-up of the merging process. 4 droplets are sorted by their fluorescent signature (pseudocolored) and directed to the DEP trap for merging (v). As the droplets encounter the actuated trap, they are sequentially merged into the assembled droplet (vi-vii). The electrode is then temporarily turned off so the assembled droplet may be released and recovered downstream (viii).

FIG. 5 provides a schematic flow diagram of a method of selectively combining discrete entities using a microfluidic device according to embodiments of the present disclosure.

FIG. 6 provides a schematic showing example configurations for trapping a discrete entity. Panel i) shows a bipolar electrode pair embedded in the same side wall of a channel. Panel ii) shows a bipolar electrode pair embedded on opposite sides of channel. Panel iii) shows bipolar electrode pair embedded in the floor or ceiling of a channel.

FIG. 7 provides a schematic showing example configurations for directing discrete entities to a discrete entity merger region. Panel i) shows application of a lamination flow to confine the laminar flow containing the droplet to the side wall of the channel. Panel ii) shows a partial height flow divider that allows fluid, but not droplets to enter the center portion of the channel. Panel iii) shows a configuration where a groove of similar height to the droplet dimensions is patterned near the side wall of a channel, while the rest of the channel is constructed with a reduced height to exclude droplets. Panel iv) shows a porous flow divider that allows fluid, but not droplets to enter the center portion of the channel. Panel v) shows a partial height flow dividers that direct droplets to a trap at the center of the microfluidic channel.

FIG. 8 provides a schematic showing an example embodiment wherein trapping is facilitated by a mechanical valve. Panel i) shows an initial stage where the discrete entities are trapped by the valve. Panel ii) shows a second stage wherein the discrete entities have been combined, e.g. due to electrical, chemical, or other means. Panel iii) shows a third stage where the combined discrete entity is released by opening the valve and carried downstream.

FIG. 9 provides a schematic showing example embodiments with different channel geometries in proximity to an electromagnetic trapping element. Panel i) shows a discrete entity merger region upstream of a bend in the channel wall. Panel ii) shows a discrete entity merger region in a lateral facet in the channel wall. Panel iii) shows a discrete entity being trapped in a region that is vertically taller than the main channel.

FIG. 10 shows the current standard workflow in the art for the identification and administration of patient specific neoantigen-based therapies. The steps in red boxes represent a bottleneck in the process.

FIG. 11. A: Workflow schematic. B and C: Wash-free immunoassay to detect Interleukin 2 released from single-cells.

FIG. 12. Cytotoxicity profiling of CAR-T-cells in isolated cell-cell interactions. A) Workflow schematic. B) Representative brightfield and fluorescent images of MOD assembled droplets. C) Cell death quantification at 3 and 12 hours for homotypic RAJI pairings and CAR-T RAJI pairings.

FIG. 13. Workflow schematic for exemplary molecular biology methods. A) Hydrogel-bound BTSOs are released by UV exposure. B) RT reaction inside droplet followed by emulsion breaking to recover cDNA. C) Nested PCR with primers complementary to BTSO and constant region of TCR α and β to specifically amplify TCR sequences with Illumina sequencing adaptors. D) TCR amplicon library is sequenced with pair-end Illumina sequencing followed by barcode and TCR sequence extraction.

FIG. 14. The MOD platform integrates three technologies for the intricate profiling of cell-cell interactive events. 1. Deterministic single-cell and reagent droplet sorter and assembler. Cells and reagents are pre-encapsulated within individual droplets, the desired final droplet contents are defined, and thousands to millions of iterations of the desired droplet are assembled. 2. Single cell-cell interaction events are profiled within individual droplets. 3. mRNA corresponding to neoantigens and their cognate TCRs are identified for cells activated during cell-cell contact.

FIG. 15 shows an exemplary embodiment of overlap PCR according to certain embodiments.

FIG. 16 shows an exemplary flow chart for methods of manufacturing CAR-T cells that are IFN-γ positive and/or IL-6 negative and using such cells for treating a patient (e.g., with cancer).

FIG. 17 shows cluster analysis results from Example 2. Over 20000 droplets were assembled in a 1:1 ratio to contain a single patient CAR-T cell and a target RAJI cell and merged with IFN-γ assay reagents in droplets. Droplets were incubated for 12 hours at 37° C. in an incubator (5% CO2). Assayed droplets were sorted for IFN-γ signal, and ˜2000 droplets were collected for the sorted AND waste.

FIG. 18 shows sorting gate results from Example 2. The gates used during sorting are depicted for both patients. Gray points indicate assembled droplets that were not sorted, whereas red points were sorted and collected. Dark red points indicate likely true hits whereas light red points indicate droplets that likely include false positives. Lenient gates were set to ensure sufficient drops were collected and to minimize the number of false negatives.

FIG. 19a shows polyfunctional gene expression cluster results from Example 2. SORT: Droplets sorted based on IFN-γ protein expression also show high levels of IFN-γ mRNA whereas droplets that end up in the WASTE do not show detectable levels of IFN-γ mRNA, which suggests that the IFN-γ based cell sorting works as intended. Cluster 3 in SORT contains CAR-T cells that share barcodes with the RAJI cells (bottom panel), which suggests that these cells possibly were processed as doublets (indicative of immune synapse) in the 10× Genomics system. The CAR-T cells associated with this cluster also are not polyfunctional suggesting potential T-cell exhaustion. FIG. 19b shows polyfunctional gene expression cluster 4 results from the waste sample from Example 2. WASTE: Cluster 6 in Waste contains cells that show polyfunctionality but IFN-γ gene expression was not detected in any of the cell populations in Waste. The remaining T-cell barcodes could be generated from un-transduced T-cells or they could be cell doublets as indicated by RAJI-specific marker genes, CD19 and HLA-DOA. Note cluster 6 does not show very few cell barcodes highlighted by CD19 and/or HLA-DOA indicating that cluster 6 indeed contains T-cells that are polyfunctional.

FIG. 20 shows an exemplary flow chart for methods of manufacturing CAR T-cells that are polyfunctional.

FIG. 21 shows construction of 20-cell spheroids using deterministic assembly as described in Example 3. A) The assembler device used to assemble 20 individually selected droplets. The microchannel in front of the trap on the right-hand side of the image is 150 um tall to accommodate larger assembled droplet volumes, with the rest of the architecture being 40 um tall. B) Assemblies at 0 hour time point show non-aggregated cells. C) Close up of an individual droplet at the 16 hour time point shows that cells have condensed into spheroids.

FIG. 22 shows the use of a coalescence sorter for single droplet sorting as described in Example 4. A) Droplets introduced through the emulsion inlet encounter an electrically charged aqueous stream, inducing merging of droplets into the stream and the exit of their contents through the aqueous outlet. B) When a droplet of interest is detected, the charge on the aqueous stream is briefly suspended, enabling the droplet to bypass the aqueous stream and exit the emulsion outlet. Droplet assembly architecture may be placed downstream of the emulsion outlet.

FIG. 23 shows a schematic of an exemplary droplet-based clonal expansion workflow that can be used upstream of droplet assay construction (e.g., as described herein with the microfluidic methods and devices). Single cells are isolated in droplets, then converted to gel beads that allow extended culturing in media while keeping groups of proliferating cells localized. After sufficient expansion has occurred, the gel beads are re-encapsulated in droplets and the gels are broken down. Finally, assays are created by assembling clonal populations of cells and assay reagents in combined assay droplets.

### DETAILED DESCRIPTION

The present invention provides compositions, systems, kits, and methods for analyzing the interaction of T-cells and neoantigen presenting cells via discrete entity (e.g., droplet) microfluids. In certain embodiments, a microfluidic device is used to merge a discrete entity containing a T-cell, and a discrete entity containing a neoantigen presenting cell, at a merger region via a trapping element in order to generate a combined discrete entity. In particular embodiments, at least one thousand of such combined discrete entities are formed in about one second. In some embodiments, whether the receptor on the T-cell sufficiently binds the neoantigen to activate the T-Cell is detected (e.g., via detection of cytokine release, granzyme B release, or CD107a release).

In certain embodiments, an approach to identify neoantigen-TCR pairs is generally composed of at least 3 steps: 1) Neoantigen discovery efforts that not only screen hundreds to thousands of neoantigens but also qualify a subset as immunogenic neoantigens, 2) Identifying the cognate TCRs for immunogenic neoantigens and 3) Database construction of functional neoantigen-TCR combinations from Human Leukocyte Antigen (HLA)-matched cancer patients. To date, neoantigen discovery efforts have gained some traction, but no high throughput methods exist that can determine the sequence of cognate TCRs. Consequently, there is no patient-centric database of matched neoantigens and TCRs.

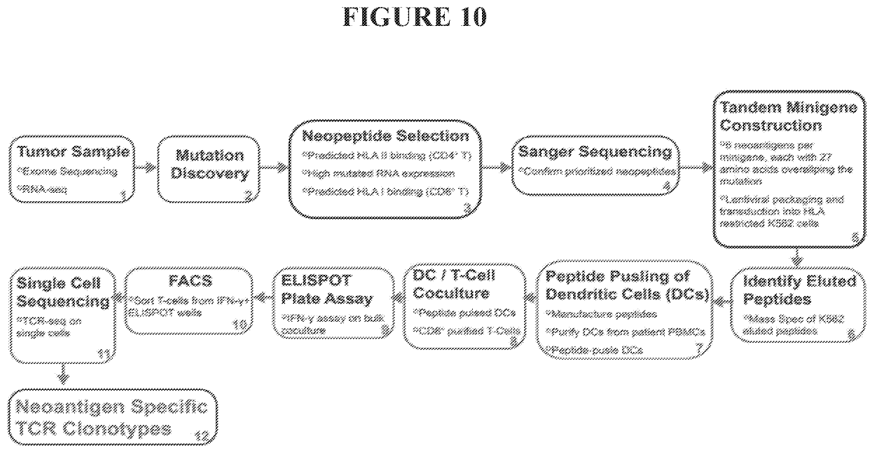

In the art, qualification of neoantigens and identifying neoantigen-specific TCRs is a multistep, laborious, time consuming and expensive process [8], [9] (FIG. 10). For a genomic aberration or posttranslational modification (PTM) to qualify as a neoantigen, the epitope has to be expressed and presented on one of the patient's HLA molecules, preferably only on the tumor cell surface, and recognized by a subset of the patient's T-cell repertoire. However, this process is highly stochastic and not all changes are immunogenic. Frequently, immunogenic changes are passenger mutations incidental to cancer progression that are unique to each patient. In addition, subsets of potential neo-antigens are expressed in individual cancer cells generating large intra-tumor heterogeneity (ITH) in variations. All of these variables have confounded neoantigen prediction algorithms. Bulik-Sullivan et. al., [10] recently reported an HLA class I prediction algorithm (step 3, FIG. 10) that significantly improves the ability to predict potential neoantigens. Other high-level steps of the workflow are shown in FIG. 10.

In reference [11], the workflow shown in FIG. 10A was used to identify neoantigens from two patients who showed durable response for cell therapy. In this approach, predicted neo-antigens for each mutation from two patients (71 mutations converted to 12 minigene clusters for patient 1 and 271 mutations converted to 37 minigene clusters for patient 2) were subcloned as tandem minigenes ([11]). Patients were also HLA-typed, and the cDNA for a specific HLA allele was co-transfected into COS7 cells. The COS7 cells are expected to present the neoantigens on HLA on the cell surface for interaction with patient T-cells. These COS7 cells were cocultured with individual patient T-cells and T-cell specific interferon-gamma (IFN-γ) secretion was assessed to identify the minigene cluster that elicited robust IFN-γ signal. Each minigene cluster comprises identified mutations from four to eight genes in tandem. To identify the individual neoantigens that patient T-cells bind to, wild type and mutant versions of the minigenes were systematically tested in a coculture assay with patient T-cells. For example, wild type KIF2C specifically abrogated the interferon-gamma response of patient T-cells. Wild type versions of potential neoantigens for the other genes in the minigene cluster did not impact IFN-γ signal in the coculture assay which suggests patient T-cell repertoire contains T-cells that kill mutant KIF2C tumor cells. This approach, while successful, is resource and time intensive and is low throughput.

The microfluidic platform and associated methods herein addresses many of the disadvantages associated with the current workflow (FIG. 10) by integrating several instrument needs in a microfluidic device. Such a platform allows deterministic single-cell combinations of patient derived APCs and T-cells, sorts these cell combinations based on a T-cell signal (e.g., cytokine release, such as IFN-γ, or granzyme B release, or CD107a release), and, in certain embodiments, integrates omics readouts. Such a platform saves days (e.g., 45 days) of time and thousands of dollars per patient sample.

In certain embodiments, the microfluidic platform integrates multiple innovations to improve the efficiency of neoantigen/cognate TCR discovery by, for example: 1) encapsulating detectably labeled, HLA-typed single APCs/tumor cells and T-cells in droplets; 2) screening thousands or a million deterministically assembled droplets; 3) performing droplet-based cytokine (e.g., IFN-γ, granzyme B, CD107a) assays, 4) sorting droplets based on cytokine signal (e.g., IFN-γ signal), 5) merging sequencing and purification nucleic acid sequences (e.g., oligo-dT beads) with cytokine (e.g., IFN-γ), granzyme B, or CD107a positive droplets and capturing mRNA from lysed cells and 6) processing neoantigen/TCR from positive (e.g., IFN-γ-positive) droplets for next-generation sequencing (NGS).

In certain embodiments, the microfluidic device comprises a microenvironment on Demand (MOD) device, described in U.S. Provisional application Ser. No. 62/847,791, which is incorporated by reference herein. In general, the MOD platform is composed of an combination of three technologies: 1) a deterministic single-cell droplet sorter and droplet-assembler that can selectively assemble cells and reagents 2) cell-based assays adapted to single-cell combinations in droplets and 3) molecular biology methods that can capture mRNAs corresponding to neoantigens and their cognate TCRs and process them for DNA sequencing.

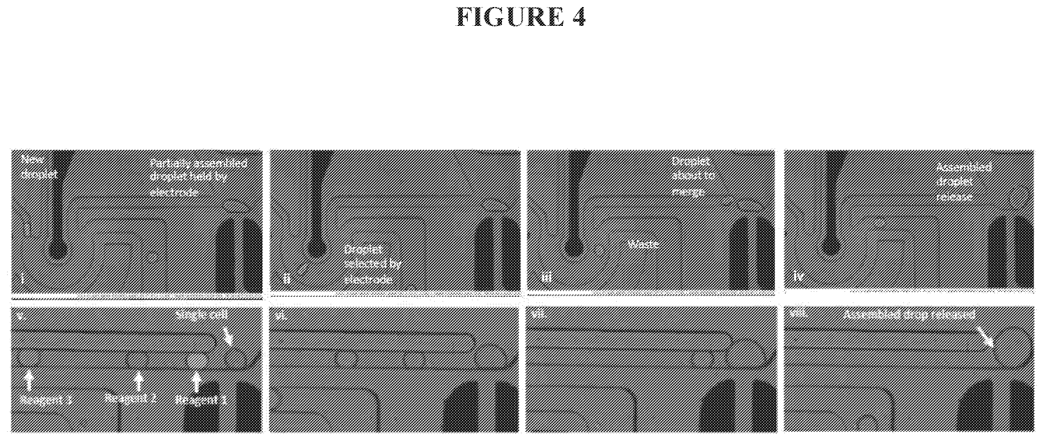

MOD performs a cyclic buildup and release of designer droplets through the merging of select droplets on a defined dielectrophoretic trapping position inside the microfluidic device (FIG. 4). This approach is advantageous because it is less prone to contamination, higher throughput, and requires fewer moving parts than other devices. The flexible nature of the MOD platform makes it a well-suited technology to perform integrated and functional cell-cell, cell-ECM interaction analysis and link any perturbations to select expressed gene sequences or transcriptome profiles at a single cell level. Essentially, MOD allows for precise, flexible, scalable liquid handling that can build a large number of predetermined reaction conditions.

MOD not only allows for the sorting and combination of particulates (cells, beads, hydrogels, etc.), but also sort and assemble diverse droplet contents (e.g., antibody solutions, cell stains, oligonucleotides etc.). Furthermore, droplet experiments constructed with MOD are compartmentalized and miniaturized (e.g., ˜100 pL) providing contained reactions in concentrated volumes. These two aspects of MOD, reagent selection and reaction miniaturization, provide a powerful approach to phenotypically screen large numbers of single cells.

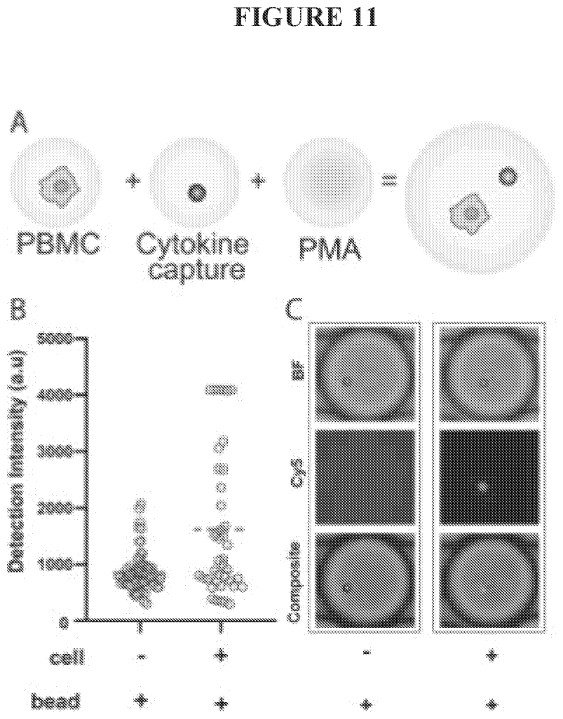

The secretion of inflammatory cytokines (e.g., TNFα, IL-2, IFNγ etc.) or granzyme B or CD107a can be used to identify activated T-cells using the MOD platform. An indrop assay can be used to detect activated cells through cytokine detection. Such an assay quantifies cell-secreted cytokine concentrations from individual cells within individual droplets (See, FIG. 11, A). The readout relies on a sandwich immunoassay, similar to ELISA, but in a “one-pot” format that is suitable for droplets and without washing steps. Activatable cells (T-Cells), an activating stimulus (phorbol myristate acetate or APC), microparticles coated with a capture antibody, and fluorescently labeled detection antibody are assembled in droplets using MOD. Following a short incubation and activation period, secreted cytokine present within droplets is sandwiched between the capture microparticle and fluorescent reporter. The cytokine concentration may then be determined by the fluorescent relocation of secondary antibody from the droplet media to the bead.

In work conducted during development of embodiments herein, such an assay was used to identify activated cells from a diverse population. FIG. 11 highlights the detection of activated T-cells based on IL-2 secretion, from PBMCs following non-specific stimulation with PMA. Other work evaluated activation following a cell-cell interaction event using T-cells with a chimeric antigen receptor (CAR-T) that activates in the presence of CD19+ RAJI cells. It was found that that fluorescent relocation provides an identifiable sorting signal with utility for binary sorts of activated vs non-activated cells.

In certain embodiments, T-cell activation is detected by detecting activation molecule release from said T-cells (e.g., ATP, cytokines, granzyme B, and CD107a). In particular embodiments, reagents for detecting such activation molecules are included in a discrete entity or combined discrete entity. In certain embodiments, the detection reagents are on beads. In other embodiments, the detection reagents are in solution, not attached to surface. Examples of such solution based reagents and detection are aptamer-based detection, proximity assays, and fluorogenic or activatable small molecule enzyme substrates.

Aptamer-based approaches typically rely on using a modified or unmodified nucleic acid that binds specifically to the target of interest (e.g. protein or small molecule, such as ATP, cytokines, granzyme B, and CD107a). Upon binding, the state, local environment, or structure of the aptamer changes allowing either direct detection, or amplification of the bound aptamer prior to detection. Binding of the aptamer to target can also release a hybridization partner (e.g. a complementary oligo or small molecule). Detection can be performed via fluorescence, absorbance, or quantification of the aptamer or released hybridization partner (see, e.g., Xue, L., et al. (2012). “Sensitive and homogeneous protein detection based on target-triggered aptamer hairpin switch and nicking enzyme assisted fluorescence signal amplification.” Anal Chem 84(8): 3507-3513; herein incorporated by reference).

Proximity assays rely on the interaction of two binding partners brought into close proximity by the target molecule (e.g., ATP, cytokines, granzyme B, and CD107a). Binding partners are typically nucleic acids or protein fragments. Binding partners can be bound directly or indirectly to detection reagents, such as antibodies or aptamers, or bind the target directly. When multiple detection reagents (with interaction partners bound to them) are both bound to the target, or when multiple interaction partners bind the target directly, the close proximity of the attached binding partners allows for covalent linkage (e.g. via ligation), hybridization, or general interaction. The interaction of the binding partners allows for detection via quantification of the bound partners, amplification of bound partners, or direct measurement using for example fluorescence (See, e.g., Xiao, Q., et al. (2018). “Multiplexed chemiluminescence imaging assay of protein biomarkers using DNA microarray with proximity binding-induced hybridization chain reaction amplification.” Anal Chim Acta 1032: 130-137; herein incorporated by reference in its entirety).

Fluorogenic and other activatable small-molecule detection strategies rely on direct modification of a substrate by the target of interest. Detection is performed directly on the modified substrate using for example fluorescence or absorbance. In certain embodiments, T-cell activation is based on granzyme B substrate cleavage detection. In some embodiments, a granzyme B substrate is included in a discrete entity or combined discrete entity. Examples of such granzyme B substrates includes, but are not limited to, Ac-IETD-AFC, Ac-IEPD-AMC, Ac-IETD-pNA, and Ac-IEPD-pNA.

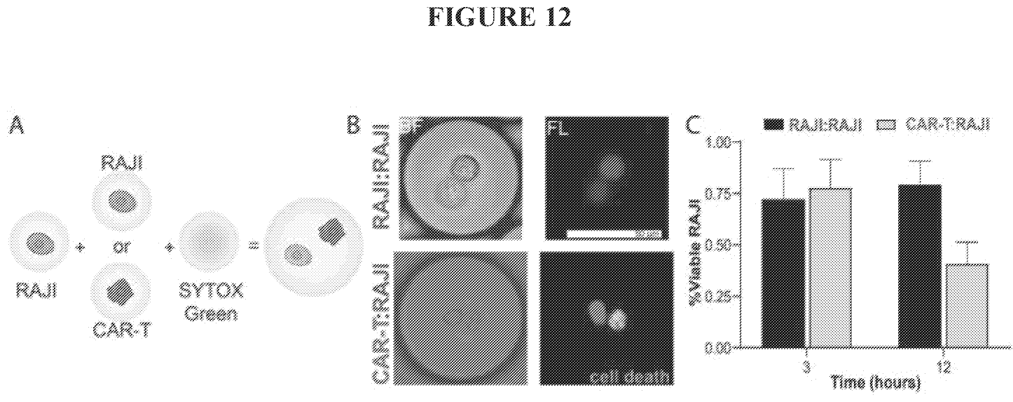

In addition to T-cell activation, work conducted during development of embodiments herein has shown how MOD allows for cytotoxic profiling of cellular combinations. Cell-death assays were performed by selectively pairing two cells within assembled droplets and incubating for 12 hours in the presence of a fluorescent-cell death stain (SYTOX® Green) (FIG. 12). Single αCD19 CAR-T-cells and target RAJI cells were paired to form experimental droplets, and homotypic pairings of RAJI cells were constructed as control droplets. At 3 and 12 hours RAJ cell death was analyzed via fluorescence. No observed decrease in RAJI viability was observed for control droplets between 3 and 12 hours. Conversely, RAJI viability decreased from 78% at 3 hours to 41% at 12 hours when paired with a CART-cell. This decrease in viability in a subset of droplets is consistent with typical human ratios of CD4+/CD8+ Tcells as only the cytotoxic CD8+ T-Cell subset of CAR-T-cells are expected to directly kill target-cells. Similar to cytokine detection, the fluorescent signature of dead cells provides a signal that can used for droplet sorting.

In some embodiments, the MOD platform couples cell-based assay results to sequencing readouts. Such coupling is used to obtain matched neoantigen-TCR sequence informatics. For example, incubated droplets containing APCs/T-cells/IFN-γ beads can be assayed as described above, sorted for activated T-cells based on IFN-γ signal (or other cytokine signal), and processed for next generation sequencing (NGS). The variable regions are encoded in the 5′ end of the TCR α and β subunit genes. The minigene clusters (e.g., if employed for the neoantigens) that encode up to 6 minigenes are expected to be ˜600 bp in length. Therefore, one exemplary strategy to sequence the variable regions of the TCR α and β subunit genes as well as the cognate minigenes is to barcode the cDNA synthesized from each droplet at the 5′end and use 300 bp paired end sequencing.

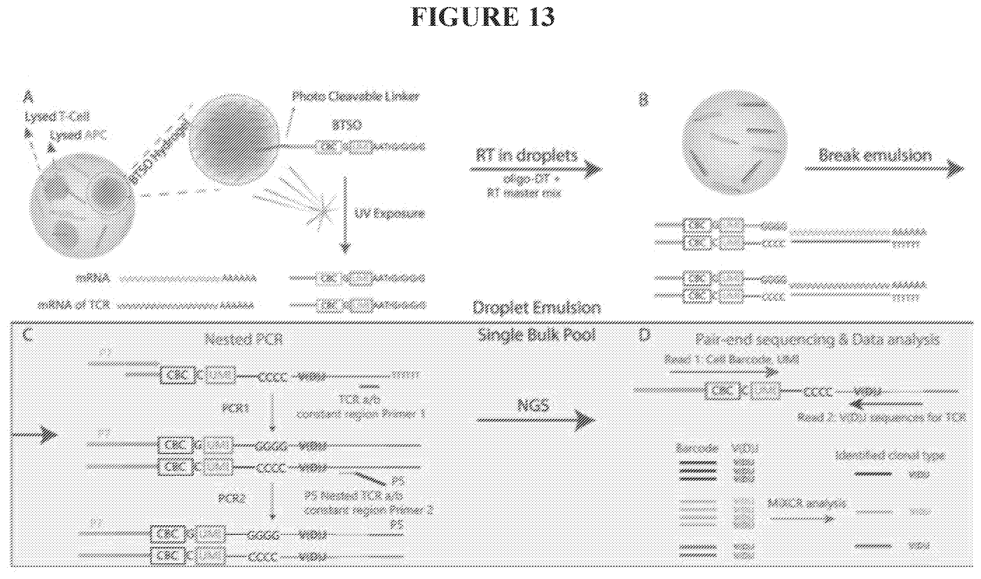

An exemplary strategy for sequencing IFN-γ positive droplets is shown in FIG. 13. Cell-pair containing droplets are merged with droplets containing lysis buffer and lysed. Hydrogel microspheres carrying barcoded template switch oligos (BTSOs) with photocleavable linkers and reverse transcriptase (RT) master mix (contains oligo dTs) are encapsulated in droplets and combined with droplets containing lysed cells [18]. Template switching mechanism of reverse transcriptase adds non-templated Cs to the ends of synthesized cDNA to which BTSOs hybridize using their riboG overhangs and continue to synthesize the complement of the BTSO sequence, thereby tagging each cDNA molecule in each droplet with a unique barcode. The droplets are collected in a tube and incubated for 60-90 min at 42° C. The emulsions are broken and the synthesized cDNAs are used as template to perform nested PCR using TCR α and β subunit and neoantigen specific 3′ primers and 5′ P7 Illumina adapter primers. To obtain transcriptomic profiles, the cDNA template is split in two after emulsions are broken and one part will be used for transcript profiles and the other is used for sequencing variable regions of TCR and its cognate neoantigen.

In general, the approach described herein allows for thousands or millions of experiments screening libraries of TCRs against peptide:MHC conjugates. Exemplary advantages of this approach are as follows. First, querying larger numbers of cells allows access to a significant subset of the TCR-antigen interaction space which can span 100,000s of TCRs and 1000s of antigens. As this space is more comprehensively mapped out, this information can, in some embodiments, be used to inform more generalized treatments. Second, the ability to build cell interaction experiments with single cell resolution enables more reproducible stimulation of T-cells by APCs than the uncontrolled local conditions in bulk experiments. Third, by redundantly encoding potential antigens to several different gene clusters transfected into APCs, a comparison of assay positive experiments allows the precise identification of the TCR-activating peptide without an additional cycle of knock out experiments.

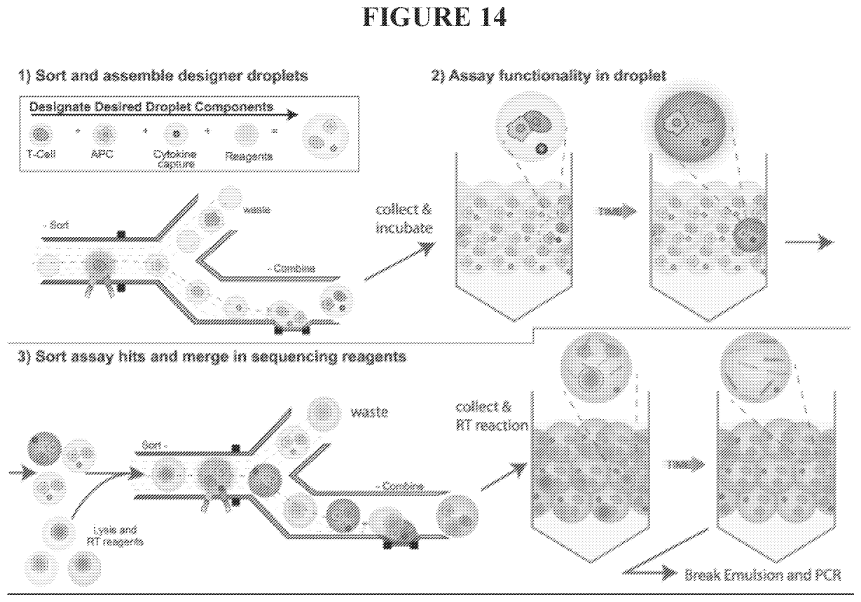

In certain embodiments, the MOD platform will input T-cell and APC (antigen-presenting cell) libraries as shown in FIG. 14. As shown in FIG. 14, the MOD platform provides, for example, performing 4 color fluorescence detection and real-time droplet assembly on a chip containing microfluidics.

An exemplary embodiment of employing the MOD platform for T-cell-APC co-culture, and detecting T-cell activation with less than 24 hour incubation period is as follows. HLA-A2 transfected K562 cells are pulsed with NY-ESO-1 peptides in bulk culture. Peptide-pulsed K562 cells, anti-NY-ESO-1 T-cells, and IFNy assay beads are fluorescently labeled, then encapsulated within individual microfluidic droplets as single cells or single beads. This mixed emulsion is run through the MOD platform such that each assembled droplet contains one T-cell, one K562 cell, and one assay bead. These assembled droplets are collected in an external Eppendorf tube and incubated for 4-24 hours, with the duration of the incubation being set by the time required to generate detectable quantities of IFNy. All incubated emulsions are fluorescently imaged and analyzed for IFNy secretion. As a benchmark comparison, the same T-cells and peptide pulsed APCs are co-cultured in bulk and the T-cells are assayed for IFNy secretion via ELISPOT assay [24]. All incubated emulsions are reinjected into the MOD device and sorted based on IFNy fluorescence using the instrument's onboard fluorescence detection capabilities. Alternative biomarkers for T-cell activation, such as CD107a, are employed in certain embodiments (e.g., such that immunoassays beads are not necessarily needed).

An exemplary embodiment employing APCs with nucleic acids encoding peptide gene clusters and TCR and peptide sequencing is as follows. This embodiment allows for the testing of large numbers of antigen candidates and sequencing methods to identify TCR-neoantigen pairs. First, HLA-A2 compatible influenza and NY-ESO-1 peptides are encoded in a 6-minigene gene cluster that includes four antigenic sequences compatible with other previously characterized HLA alleles. Six minigene cluster is about ˜600 bp that can be sequenced using paired-end 300 bp sequencing in a MiSeq. This minigene cluster is packaged into lentiviral particles and transfected into HLA-A2 restricted K562 cells. Modified HLA-A2 restricted K562s are paired with anti-NY-ESO-1 T-cells and IFNy assay beads using the MOD platform, incubated, and assayed for IFNy via imaging. Methods are then employed to simultaneously sequence TCR chains and encoded peptide genes for each discrete cell combination. In certain embodiments, the HLA alleles on the APCs are also sequenced.

As shown in FIG. 14, the sequencing workflow involves injecting droplets containing cell pairs into the MOD device, selecting droplets with positive IFNy signals, and merging in RT master mix that contain oligo-dT and BTSOs. Unique BTSOs are added to each droplet by using hydrogel beads as carriers. A library of hydrogel beads are synthesized through microfluidic emulsification of hydrogel precursors and primers, gelation, and a series of split-and-pools and primer extension reactions as previously described [18]. Importantly, the resulting BTSO are bound to the hydrogel through a photocleavable linker, which allows for release of the BTSO once inside the droplet by exposure to UV light. The droplets are then be collected externally and incubated for RT, followed by emulsion breaking and processing for next generation sequencing (FIG. 13). In certain embodiments, it is not necessary to obtain paired TCR alpha chain and beta chains from every droplet. For example, it has been described that the VDJ recombined complementarity-determining region 3 (CDR3) region is sufficient to match TCR chains [see, 27, herein incorporated by reference], so adequate TCR sequence information, in certain embodiments, is available from droplets that provide only information on one TCR chain and the minigene cluster sequence.

In certain embodiments, the MOD platform is employed (e.g., in the T-cell context or general MOD platform) and droplet manipulation and sorting is be achieved by electrowetting, the modification of the wetting properties of a surface with an applied electric field. Electrowetting manipulation of droplets in a microfluidic device may be achieved through the application of differential voltages to different regions in an electrode grid (see, U.S. Pat. No. 6,911,132, herein incorporated by reference). Alternatively, droplet actuation and sorting can be achieved using opto-electrowetting, where localized electric fields are triggered through the selective application of light to a photoconductive layer (see, U.S. Pat. No. 6,958,132, which is herein incorporated by reference in its entirety).

In certain embodiments, droplet-based cell culture is performed using porous materials. The duration of cell culture in sub-nanoliter droplets is limited by a finite amount of encapsulated media and localized buildup of metabolic waste products. In cases where longer duration incubations are desired or required, it may be appropriate to convert a droplet to a media-permeable format while keeping encapsulated objects in place. This can be achieved by flowing hydrogel precursors into droplets along with cells, then triggering gelation to form either gel beads or permeable capsules. After gelation, the emulsion is broken, the emulsion oil is removed, and the cell-laden beads or capsules are suspended in media and cultured for a time. Examples of the hydrogel bead approach are given in Wan et al., (Polymers (Basel), vol. 4, no. 2, pp. 1084-1108, 2012), Utech et al., (Adv. Healthc. Mater., 2015), and Dolega et al. (Biomaterials, vol. 52, no. 1, pp. 347-357, 2015.)—all of which are herein incorporated by reference in their entireties. Examples of permeable capsules are given by Yu et al, (Biomed. Microdevices, vol. 17, no. 2, 2015.), van Loo et al (Mater. Today Bio, vol. 6, no. February, p. 100047, 2020.), and Leonaviciene et al. (Lab Chip, no. Advanced Article, 2020), all of which are herein incorporated by reference in their entireties. Extended cell culture is especially useful in cases where cell proliferation is important, such as clonal expansion of single cells and cell-cell interaction assays where proliferation is a readout. In some cases, it may be necessary to break down a gel bead or capsule via chemical, enzymatic, or thermal means in order to access the contents for further processing. In other cases, gel beads and capsules are sufficiently permeable that analysis can be performed with the materials in places, such as the washing in of assay reagents (Chokkalingam et al., Lab Chip, vol. 13, no. 24, pp. 4740-4744, 2013) or sequencing reagents (Leonaviciene).

Discrete entities as used or generated in connection with the subject methods, devices, and/or systems may be sphere shaped or they may have any other suitable shape, e.g., an ovular or oblong shape. Discrete entities may be droplets. Discrete entities as described herein may include a liquid phase and/or a solid phase material. In some embodiments, discrete entities according to the present disclosure include a gel material. In some embodiments, the subject discrete entities have a dimension, e.g., a diameter, of or about 1.0 μm to 1000 μm, inclusive, such as 1.0 μm to 750 μm, 1.0 μm to 500 μm, 1.0 μm to 100 μm, 1.0 μm to 10 μm, or 1.0 μm to 5 μm, inclusive. In some embodiments, discrete entities as described herein have a dimension, e.g., diameter, of or about 1.0 μm to 5 μm, 5 μm to 10 μm, 10 μm to 100 μm, 100 μm to 500 μm, 500 μm to 750 μm, or 750 μm to 1000 μm, inclusive. Furthermore, in some embodiments, discrete entities as described herein have a volume ranging from about 1 fL to 1 nL, inclusive, such as from 1 fL to 100 pL, 1 fL to 10 pL, 1 fL to 1 pL, 1 fL to 100 fL, or 1 fL to 10 fL, inclusive. In some embodiments, discrete entities as described herein have a volume of 1 fL to 10 fL, 10 fL to 100 fL, 100 fL to 1 pL, 1 pL to 10 pL, 10 pL to 100 pL or 100 pL to 1 nL, inclusive. In addition, discrete entities as described herein may have a size and/or shape such that they may be produced in, on, or by a microfluidic device and/or flowed from or applied by a microfluidic device.

In some embodiments, the discrete entities as described herein are droplets. The terms “drop,” “droplet,” and “microdroplet” are used interchangeably herein, to refer to small, generally spherically structures, containing at least a first fluid phase, such as an aqueous phase (e.g., water), bounded by a second fluid phase (e.g., oil) which is immiscible with the first fluid phase. In some embodiments, droplets according to the present disclosure may contain a first fluid phase (e.g., oil) bounded by a second immiscible fluid phase (e.g., an aqueous phase fluid, such as water). In some embodiments, the second fluid phase is an immiscible phase carrier fluid. Thus, droplets according to the present disclosure may be provided as aqueous-in-oil emulsions or oil in aqueous emulsions. Droplets may be sized and/or shaped as described herein for discrete entities. For example, droplets according to the present disclosure generally range from 1 μm to 1000 μm, inclusive, in diameter. Droplets according to the present disclosure may be used to encapsulate cells, nucleic acids (e.g., DNA), enzymes, reagents, and a variety of other components. The term droplet may be used to refer to a droplet produced in, on, or by a microfluidic device and/or flowed from or applied by a microfluidic device.

As used herein, the term “dielectrophoretic force” refers to the force exerted on an uncharged particle caused by of the polarization of the particle by and interaction with a nonuniform electric field. A dielectrophoretic force can be directed towards (i.e. “attractive dielectrophoretic force”), away from (i.e. “repulsive dielectrophoretic force,”) or in any direction relative to the source of the electric field. Before being contacted by the electric field, the particle can be positively charged, negatively charged, or neutral.

As used herein, the term “electrophoretic force” refers to the force exerted on a charged particle caused by interaction with an electric field. An electrophoretic force can be directed towards (i.e. “attractive electrophoretic force”) away from (i.e. “repulsive electrophoretic force,”) or in any direction relative to the source of the electric field. Before being contacted by the electric field, the particle can be positively charged, negatively charged, or neutral.

As used herein, the term “carrier fluid” refers to a fluid configured or selected to contain one or more discrete entities (e.g., droplets) as described herein. A carrier fluid may include one or more substances and may have one or more properties (e.g., viscosity), which allow it to be flowed through a microfluidic device or a portion thereof. In some embodiments, carrier fluids include, for example: oil or water, and may be in a liquid or gas phase.

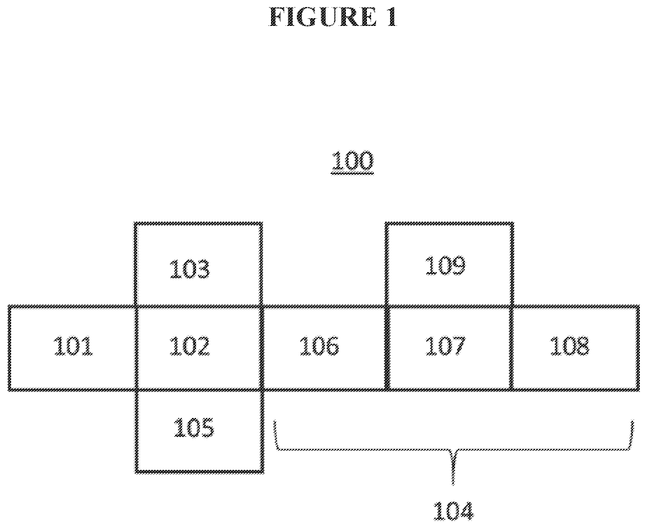

The present disclosure provides methods of selectively combining discrete entities using the MOD platform. FIG. 1 presents a non-limiting, simplified, schematic representation of one type of device and method according to the present disclosure. The microfluidic device of FIG. 1 is labeled as microfluidic device 100. FIG. 1 shows a representation of an inlet channel 101, wherein a discrete entity that is insoluble and/or immiscible in a carrier fluid a carrier fluid can be flowed through the inlet channel 101 to a sorter channel 102 that is in direct fluid communication with inlet channel 101. Next, the discrete entity can be sorted into a first outlet channel 104 or a second outlet channel 105, which are both in direct fluid communication with the sorter channel, by sorting element 103. Sorting element 103 can be, in some cases, an electrode, such as an electrode that is configured to exert a dielectrophoretic force on the discrete entity. Sorting element 103 in FIG. 1 is configured to sort a discrete entity in sorting channel 102 to first outlet channel 104 or second outlet channel 105. In some cases, if the discrete entity is sorted to second outlet channel 105, the discrete entity is sorted to a waste container or is recycled back to inlet channel 101. FIG. 1 shows an embodiment wherein first outlet channel 104 includes an upstream region 106, a discrete entity merger region 107, and a downstream region 108. In some cases, the discrete entity merger region comprises a change in a dimension of the first outlet channel, such as where the discrete entity merger region 107 has a larger cross-sectional area than the upstream region 106.

In addition, the FIG. 1 device includes trapping element 109. In some cases, trapping element 109 includes a trapping electrode, and the trapping electrode is configured to exert a force (e.g. a dielectrophoretic force), that traps the discrete entity in the discrete entity merger region 107. Furthermore, the discrete entity merger region 107 and the trapping element 109 are configured such that a force applied by the trapping electrode in the discrete entity merger region is sufficient to trap a plurality of discrete entities in the discrete entity merger region for a time sufficient for the plurality of discrete entities to combine to form a combined discrete entity. In some cases, a trapping electrode is configured to provide an electric field that affects the surface of the discrete entities such that the discrete entities can more easily merge (e.g. the discrete entities will spontaneously merge). In some cases, the affecting is destabilizing.

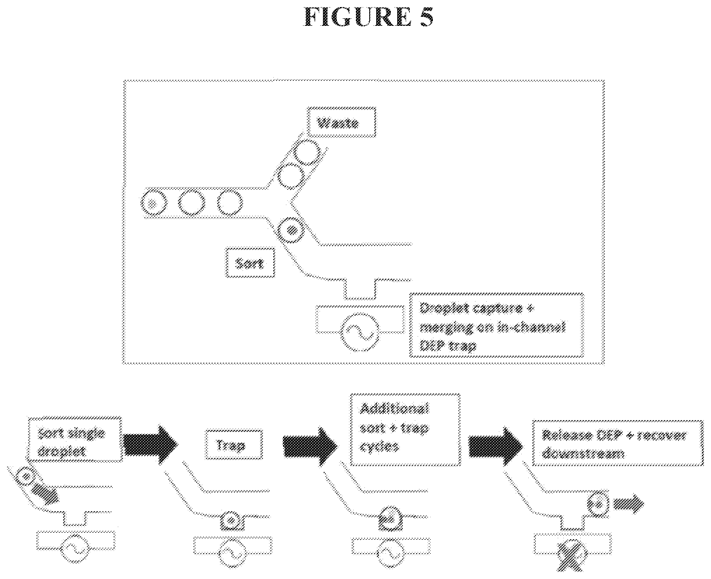

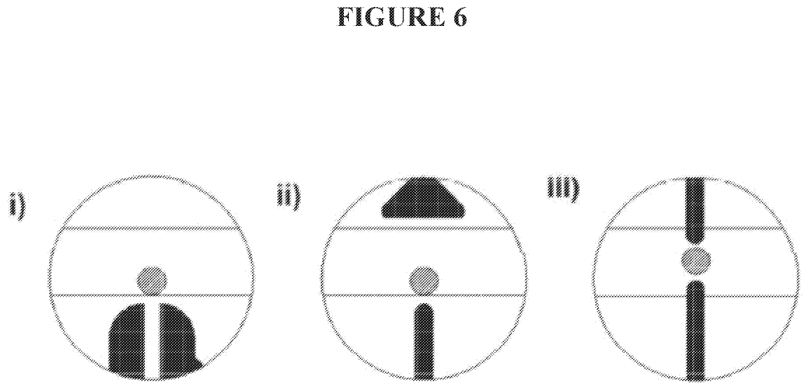

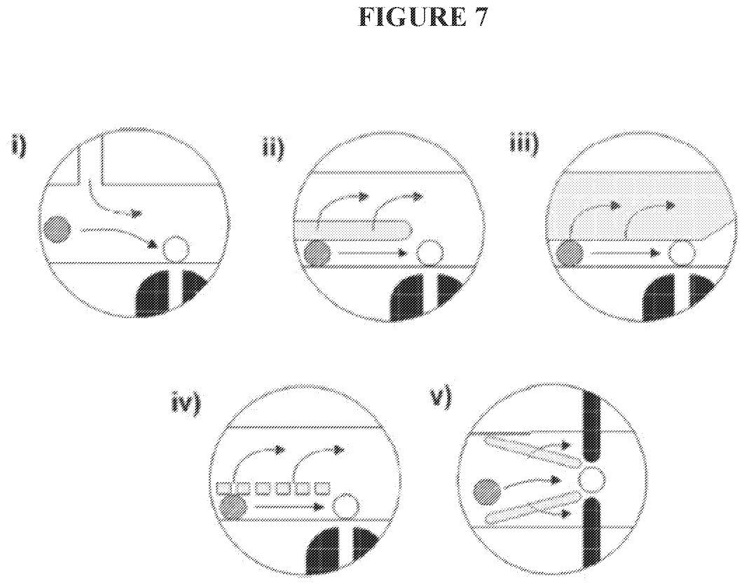

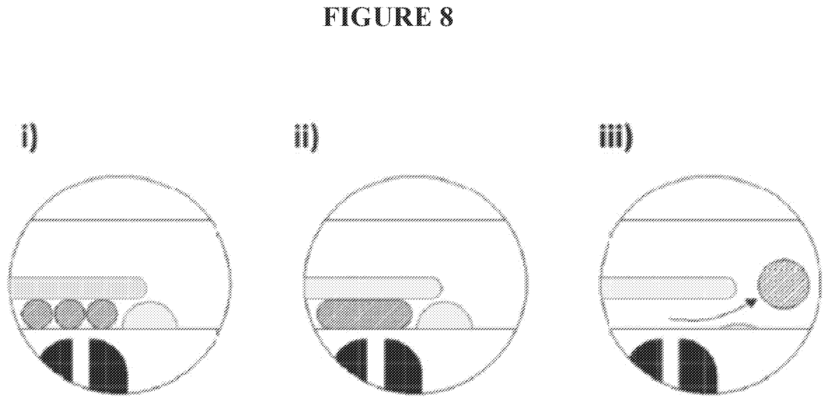

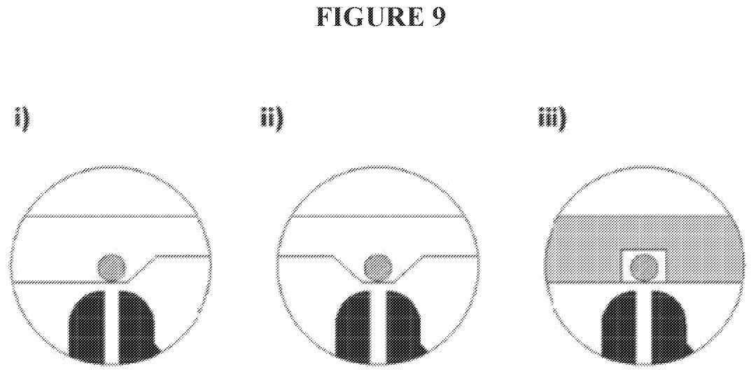

Methods of using the FIG. 1 device include flowing a plurality of discrete entities through inlet channel 101 to sorting channel 102, sorting with sorting element 103 the plurality of discrete entities into first outlet channel 104 or second outlet channel 105, trapping with trapping element 109 at least two discrete entities (e.g., one with a T-cell and one with an antigen presenting cell, presenting a neoantigen) in discrete entity merger region 107 for a time sufficient for the at least two discrete entities to combine to form a combined discrete entity. FIG. 5 shows a schematic representation of an exemplary method wherein discrete entities containing cells are selectively combined.

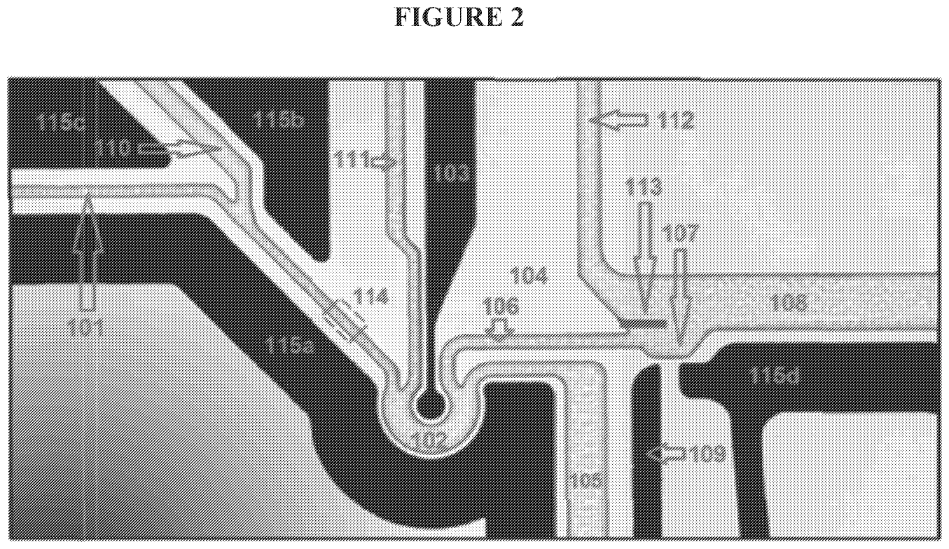

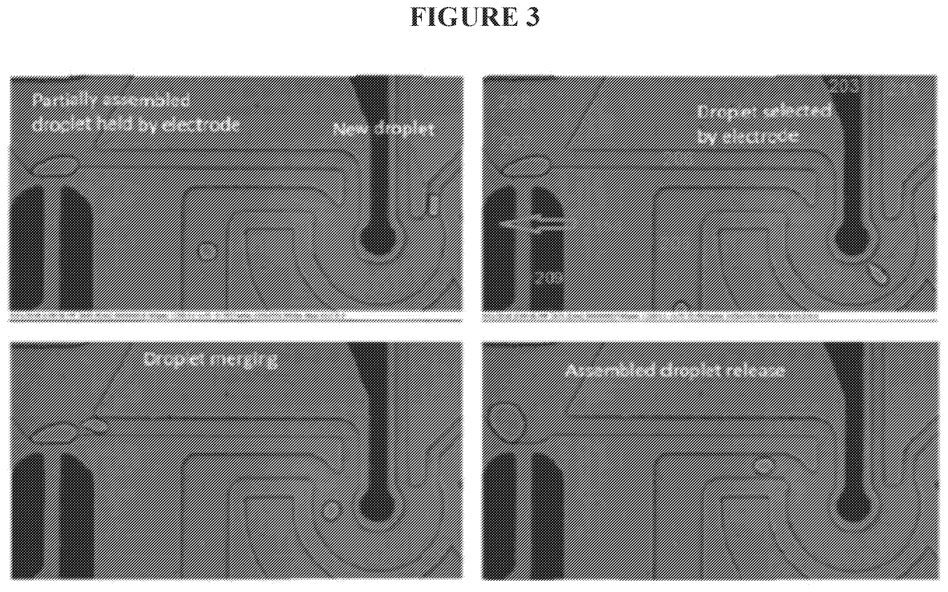

FIG. 2 presents an additional, non-limiting, simplified, schematic representation of one type of a device and method according to the present disclosure. In some cases, the discrete entity merger region includes a recess, such as shown as recess 107 in FIG. 2. In some cases, the discrete entity merger region includes a flow divider, such as shown as flow divider 113 in FIG. 2. In some cases, the device further includes a laminating oil inlet, such as shown as laminating oil inlet 112 in FIG. 2. In some cases, the trapping element includes two electrodes that have a significantly different shape from one another, such as shown as electrodes 109 in FIG. 2. In some cases, the trapping element includes two electrodes that produce a region of high electric field gradients that extends into the microfluidic channel. In some cases, the discrete entity merger region includes a change in the angle of flow between an adjacent upstream region and the discrete entity merger region, e.g. as shown in FIG. 3. In some cases, the device further includes a spacer fluid inlet. As an example, the device in FIG. 2 includes spacer fluid channel 110 in fluid communication with the inlet channel 101. The spacer fluid channel can be configured such that flowing spacer fluid through the spacer fluid channel causes spacer fluid to be located between two discrete entities flowing through the inlet channel, thereby maintaining or increasing the distance between the two discrete entities, thereby allowing each of the two discrete entities to be independently sorted or not sorted.

In some cases, the device further includes a bias fluid inlet. As an example, the device in FIG. 2 includes bias fluid channel 111 in fluid communication with sorter channel 102. The bias fluid channel can be configured such that flowing bias fluid through the bias fluid channel will cause a discrete entity to move closer to a second side wall of the sorter channel and farther away from a first side wall of the sorter channel. Thus, as an example, the spacer fluid inlet 111 would cause the discrete entity to move closer to the wall of the inlet channel that is closer to the bottom of the figure, and further away from the wall closer to the top of the figure. As such, one or more bias fluid channels can be configured such that a discrete entity will preferentially flow to a first outlet location or a second outlet location in the absence of a force from a sorting element. In some cases, the bias fluid inlet channel can be configured such that a discrete entity will preferentially flow to a second outlet channel in the absence of a dielectrophoretic force from a sorting electrode. As an example, the bias fluid inlet 111 in FIG. 2 causes a discrete entity to preferentially flow to second outlet channel 105 in the absence of a force exerted on the discrete entity by the sorting electrodes 103.

In some cases, the device includes a detector configured to detect a discrete entity in the input channel, wherein the microfluidic device is configured to sort a discrete entity in the sorting channel based on the detection by the detector. As an example, FIG. 2 shows an embodiment in which a discrete entity in detection region 114 of inlet channel 101 can be detected by a detector, after which sorting electrodes 103 can sort the discrete entity into the first outlet channel 104 or the second outlet channel 105. The FIG. 2 devices also includes shielding electrodes 115a, 115b, 115c, and 115d. As used herein, the term “shielding electrode” is used interchangeably with “moat electrode.” Each shielding electrode can be configured to perform one or more functions including: at least partially shielding discrete entities from undesired electromagnetic fields, assisting with the sorting of discrete entities, and assisting with the trapping of discrete entities.

As such, as used herein, shielding electrodes can also be referred to as sorting electrodes or trapping electrodes if such electrodes are configured to participate in the sorting or trapping of discrete entities. Hence, shielding electrode 115a can also be referred to as a sorting electrode if it is configured to form a bipolar electrode pair with sorting electrode 103 to facilitate the sorting of discrete entities. Similarly, shielding electrode 115d can also be referred to as a trapping electrode if it is configured to form a bipolar electrode pair with trapping electrode 109 to facilitate the trapping of discrete entities.

In some cases, a shielding electrode can generate an electromagnetic field such that discrete entities in the device is at least partially shielded from undesired electromagnetic fields. Such undesired electromagnetic fields can originate from outside the microfluidic device or from within the microfluidic device. In some cases, the undesired electromagnetic fields are those fields that are not generated by a sorting electrode or by a trapping electrode. By at least partially shielding discrete entities in the microfluidic device, the shielding electrodes can inhibit the unintended merging of discrete entities (i.e. merging of discrete entities outside the discrete entity merger region). In some cases, shielding electrodes 115a, 115b, and 115c can be used to at least partially shield discrete entities from electromagnetic fields that are not generated by the sorting electrode or the trapping electrode.

In some cases, shielding electrodes can assist with the sorting of discrete entities. As an example, shielding electrode 115a can interact with sorting electrode 103 in order to facilitate sorting, such as by forming a bipolar electrode pair with sorting electrode 103. In some cases, sorting electrode 103 can be the charged electrode (e.g. positively charged), and shielding electrode 115a can be a ground. Stated in another manner, shielding electrode 115a can be configured to influence the shape of the electromagnetic field generated by sorting electrode 103 in order to facilitate sorting.

In some cases, shielding electrodes can assist with the trapping of discrete entities. As an example, shielding electrode 115d can interact with trapping electrode 109 in order to facilitate trapping, such as by forming a bipolar electrode pair with trapping electrode 109. In some cases, sorting electrode 109 can be the charged electrode (e.g. positively charged), and shielding electrode 115d can be a ground. Stated in another manner, shielding electrode 115d can be configured to influence the shape of the electromagnetic field generated by trapping electrode 109 in order to facilitate sorting.

In some cases, one or more of the shielding electrodes are separate elements, such as when all the shielding electrodes are separate elements. In some cases, one or more of the shielding electrodes are directly electrically connected. In some cases, one or more of the shielding electrodes are different regions of a single electrode, such as part of a single piece of metal. In some cases, one or more of the shielding elements are attached to ground.

As shown in FIG. 2, in some cases, the device includes one or more shielding electrodes. In some cases, the device includes zero shielding electrodes, such as when the discrete entities are sorted using a single sorting electrode and the discrete entities are trapped using a single trapping electrode.

As such, discrete entities are sorted and selectively combined within a microfluidic device (i.e., without leaving the microfluidic device). Stated in another manner, the discrete entities are sorted and combined without leaving microfluidic sized channels and regions.

In addition, the present disclosure provides examples of specific elements and steps that can be used with the described devices, systems, and methods. As reviewed above, the trapping element and the sorting element can be electrodes that exert a dielectrophoretic force on the discrete entity. In some cases, the electrodes are microfluidic channels containing a conductive material (e.g. salt water, liquid metal, molten solder, or a conductive ink to be annealed later). In some cases, the electrodes are patterned on the substrate of the microfluidic device (e.g. a patterned indium tin oxide (ITO) glass slide). In some cases, the trapping element includes two electrodes. In some cases, the trapping element is a selectively actuatable bipolar droplet trapping electrode. In some cases, the sorting element includes two electrodes. In some cases, the sorting element includes a selectively actuatable bipolar droplet sorting electrode.

In some cases, the sorting channel includes a partial height flow divider. In some cases, the sorting channel has a concentric or essentially concentric flow path and a portion of the sorting electrode is positioned at the center of the arc of the concentric or essentially concentric flow path.

In some embodiments, the discrete entity includes a particle (e.g. a cell, such as a T-cell or APC). In some embodiments, the discrete entity includes a chemical reagent (e.g. a lysing agent or a PCR reagent). In some embodiments, the discrete entity includes both a cell and a chemical reagent. In some embodiments, the discrete entity includes a fluorescently tagged T-cell or APC.

In some cases, the sorting is passive sorting. In some cases, the sorting is active sorting (i.e., the sorting element sorts a discrete entity into one of at least two locations based on a detected property of the discrete entity or a component within the discrete entity). In some cases, the detected property is an optical property and the device further includes an optical detector (e.g. an optical detector configured to detect an optical property of a discrete entity or a component within in the inlet channel). In some cases, the optical property is fluorescence and the device further includes a source of excitation light. In some cases, the sorting is based on the detected fluorescence of a fluorescent tag on a cell in the discrete entity.

In some cases, the discrete entity merger region can include structural elements that are configured to aid in the trapping and combination of discrete entities therein. In some cases, such structural elements are configured to aid in such trapping and combining by changing the speed or direction of the flow of fluid through an area of the discrete entity merger region.

The present disclosure also provides methods of using systems that include a microfluidic device, e.g. as described above, and one or more additional components, e.g. (a) a temperature control module operably connected to the microfluidic device; (b) a detector configured to detect a discrete entity in the input channel, wherein the microfluidic device is configured to sort a discrete entity in the sorting channel based on the detection by the detector; (c) an incubator operably connected to the microfluidic device or a discrete entity maker; (d) a sequencer operably connected to the microfluidic device; (e) a device configured to make a plurality of discrete entities, wherein the device is located within the microfluidic device or separately from the microfluidic device; and (f) one or more conveyors configured to convey a particle (e.g. a cell, or a discrete entity), wherein the discrete entity can contain a particle in some cases, between any combination of: the incubator, device configured to make a plurality of discrete entities, the microfluidic device, the sequencer.

In some cases, the methods include controlling the temperature of the microfluidic device using a temperature control module operably connected to the microfluidic device. In some cases, the methods include detecting a discrete entity in the input channel of the microfluidic device (e.g. detecting an optical property of the discrete entity or a component therein), and sorting the discrete entity based on the detecting. In some cases, the method includes incubating cells in an incubator that is operably connected to discrete entity maker or a microfluidic device. In some cases, the method includes making discrete entities with a discrete entity maker, wherein the discrete entity maker is located within the microfluidic device or separate from the microfluidic device. In some cases, the method includes moving a discrete entity between components of the system (e.g. with one or more conveyors).

The present disclosure also provides steps that can be performed after the release of a combined microfluidic droplet from a discrete entity merger region. In some cases, the method includes recovering a component (e.g. a cell, a chemical compound or a combination thereof), from the combined discrete entity. In cases where a combined discrete entity includes one or more cells, the one or more cells can be analyzed (e.g. genetic information therein, such as TCR encoding sequenced, can be sequenced using a sequencer. The genetic information can include, e.g. DNA and RNA. In some cases, the sequencing includes PCR In some cases, the analysis of a discrete entity can include mass spectrometry. In some cases, the method includes printing the combined discrete entity onto a substrate, e.g. as described in US 2018/0056288, which is incorporated herein by reference for its disclosure of printing a discrete entity onto a substrate.

The present disclosure also provides a method of selectively performing reactions by selectively combining two or more discrete entities, as described above, wherein the reaction occurs between one or more components from each discrete entity. Such components can be one or more cells, one or more products derived from a cell, one or more reagents, or a combination thereof. In some cases, the one or more products derived from a cell include cell lysate, DNA, RNA, or a combination thereof. As an example, FIG. 4 shows the combination of four discrete entities, wherein three of the discrete entities each contain a different reagent and the fourth discrete entity contains a single cell (e.g., APC or T-cell). As such, FIG. 4 shows that a microfluidic device as described herein can be used to selectively combine different discrete entities, resulting in the formation of a combined discrete entity (e.g., that contains the three reagents and the T-cell and/or APC).

As such, the method of selectively performing reactions can include the combination of two or more discrete entities (e.g. three or more and four or more), which allows a T-cell and an APC cell to be brought together. In some cases, the number of discrete entities that contain at least one cell is zero discrete entities, one discrete entity, two discrete entities, or three or more discrete entities. In some cases, the number of cells in a discrete entity is one. In some cases, the method includes repeating the selective combination of discrete entities (e.g. performing the selective combination two or more times, three or more times, or four or more times, or 1000 or more times or a million or more times).

The present methods allow for the selective combination of two or more discrete entities without the need to accurately time the release or to accurate time the sorting of the two or more discrete entities. As such, in some cases, a first discrete entity is trapped in the discrete entity merger region before a second discrete entity to be combined therewith has entered the outlet channel after being sorted. In some cases, the second discrete entity has not entered the sorter channel, has not entered the inlet channel, or has not even been made when the first discrete entity is trapped in the discrete entity merger region.

The present methods allow for the sorting of discrete entities based on whether they contain a T-cell or APC, and allows the selective combination of only those discrete entities that contain the desired components. In some cases, the method involves creating 5 or more combined discrete entities per minute, including 10 or more, 25 or more, 50 or more, 75 or more, 100 or more, 150 or more, 200 or more, or 300 or more. In some cases, the method involves making 300 or more combined discrete entities per hour, including 1,500 or more, 3,000 or more, 4,500 or more, 6,000 or more, 9,000 or more, 12,000 or more, or 21,000 or more. In some cases, the sorting step is performed such that discrete entities are sorted at a rate of 0.01 Hz or more (e.g. 0.1 Hz or more, 1 Hz or more, 10 Hz or more, 100 Hz or more, 1 kHz or more, 10 kHz or more, or 30 kHz or more). In some cases, an electromagnetic sorter is used instead of a mechanical sorter (e.g. a valve, to allow for faster sorting rates). In some cases, the trapping and combining steps are performed such that a combined discrete entity is formed or released at a rate of 1 Hz or more, e.g. 10 Hz or more, 100 Hz or more, or 1,000 Hz or more.

In some cases, a discrete entity is flowed such that it reaches the discrete entity merger region between 0.1 ms to 1,000 ms after being sorted, such as between 1 ms and 100 ms, between 2 ms and 50 ms, and between 5 ms and 25 ms. In some cases, the first outlet channel is between 0.2 mm long and 5 mm long. In some cases, the first outlet channel has a dimension (e.g., width or height or diameter) of between 5 μm and 500 μm, such as between 10 μm and 100 μm. In some cases, the carrier fluid containing the discrete entities is flowed into the inlet channel at a rate of between 1 μl per hour and 10,000 μl per hour, such as between 10 μl per hour and 1,000 μl per hour, 25 μl per hour and 500 μl per hour, and between 50 μl per hour and 250 μl per hour. In some cases, the spacer fluid is injected at a rate of between 100 μl per hour and 20,000 μl per hour, such as 500 μl per hour and 5,000 μl per hour. In some cases, the bias fluid is injected at a rate of between 100 μl per hour and 20,000 μl per hour, such as 500 μl per hour and 5,000 μl per hour. In some cases, the fluid used to create cell-containing discrete entities has a concentration of between 1,000 cells per ml and 10,000,000 cells per ml, such as between 10,000 cells per ml and 1,000,000 cells per ml, and between 50,000 cells per ml and 200,000 cells per ml. In some cases, the discrete entities have a volume between 1 μl and 10,000 μl, such as between 10 μl and 1,000 μl, or between 50 μl and 500 μl.

In some cases, the one or more cells from a combined discrete entity are cultured for at least 30 minutes or more, such as 1 hour or more, 6 hours or more, 12 hours or more, 24 hours or more, 3 days or more, or 7 days or more. In some cases, the device can continuously operate by selectively combining discrete entities for 10 minutes or more, such as 30 minutes or more, 45 minutes or more, 90 minutes or more, or 180 minutes or more. In some cases, the device can make at least 100 combined discrete entities while continuously operating, such as 1,000 combined discrete entities or more, 10,000 combined discrete entities or more, or 100,000 combined discrete entities or more.

In some cases, the methods include making one or more discrete entities, such as with a discrete entity maker. In such cases, the discrete entity maker can be part of the microfluidic device or separate from the microfluidic device as otherwise described herein. If the discrete entity maker is separate from the microfluidic device, the discrete entity maker can be operably connected to the microfluidic device (e.g., such that discrete entities can flow from the maker to the microfluidic device), or the discrete entities can be moved to the microfluidic device without the discrete entity maker and microfluidic device being operably connected. The systems and devices can include one or more discrete entity makers configured to form discrete entities from a fluid stream. Suitable discrete entity makers include selectively activatable droplet makers and the methods may include forming one or more discrete entities via selective activation of the droplet maker. The methods may also include forming discrete entities using a droplet maker, wherein the discrete entities include one or more entities which differ in composition. In some cases, the discrete entity maker comprises a T-junction and the method includes T-junction drop-making. In some cases, making the discrete entities includes a step of emulsification. In some cases, the discrete entity maker is made, in part or in whole, of a polymer. In some cases, one or more surfaces of the discrete entity maker are coated with a fluorosilane (e.g. such a discrete entity maker can be used when fluorinated fluids are passed through the discrete entity maker).

In some cases when multiple types of discrete entities are made (e.g., discrete entities that contain different contents, such as one with a T-cell and one with an APC), the contents can affect the ability of the discrete entity maker to successfully make the discrete entities. As such, in some cases, different conditions for the discrete entity maker are used to make a first group of discrete entities with first contents than for making a second group of discrete entities with second contents.

Aspects of the disclosed methods may include making discrete entities using one or more cells from a biological sample. In such cases, each discrete entity may contain zero, one, or more than one cell. In some cases, such discrete entities can be made by incorporating the biological sample, cells from the biological sample, lysate from cells of the biological sample, or any other sample derived from the biological sample into a mixed emulsion. In some cases, the method further includes separating one or more components of the biological sample or otherwise processing the biological sample (e.g. via centrifugation, filtration, and the like), before making the discrete entities.

In some cases, after the making of the discrete entities but before introducing the discrete entities to an inlet channel of a microfluidic device as described herein, the discrete entities can be further modified (e.g. by adding a T-cell, APC, a reagent, a drug, a hydrogel, an extracellular matrix, a bead, a particle, a biological material, media, or a combination thereof). In some cases, the reagent is a primer, a probe, a lysing agent, a surfactant, a detergent, a barcode, or a fluorescent tag. In some cases, the bead is an RNA capture bead. In some cases, the bead is an immunoassay bead. In some cases, the barcode is an oligonucleotide. In some cases, different types of discrete entities are labeled with different types of barcodes, fluorescent tags, or a combination thereof.

Fluorescent tags can be used to image a discrete entity or combined discrete entity in the discrete entity merger region. Fluorescent tags can also be used to identify the particular type of discrete entities that were combined to create a given combined discrete entity. As such, the properties of the combined discrete entity or component thereof can be correlated with the contents that were used to make the original discrete entities. As an example, different types of T-cells can be labeled with different fluorescent tags and incorporated into discrete entities. After such T-cell-containing discrete entities are combined with other discrete entities (e.g. containing antigen presenting cells (APCs)), the outcome of the combined discrete entities can be observed (e.g., T-cell activation via cytokine release). As some of all of the original discrete entities can be labeled with fluorescent tags, the resulting combined discrete entity can have multiple fluorescent tags. In other cases, the combined discrete entity only has one fluorescent tag. Oligonucleotide barcodes can be used in a similar manner to that of fluorescent tags. Instead of detecting optical fluorescence, however, the oligonucleotide barcodes can be sequenced in order to identify the original discrete entities that formed the combined discrete entity.

Methods and devices which may be utilized in the encapsulating of a component from a biological sample are described in PCT Publication No. WO 2014/028378, the disclosure of which is incorporated by reference herein in its entirety and for all purposes. Encapsulation approaches of interest also include, but are not limited to, hydrodynamically-triggered drop formation and those described by Link, et al., Phys. Rev. Lett. 92, 054503 (2004), the disclosure of which is incorporated herein by reference. Other methods of encapsulating cells into droplets may also be applied. Where desired, the cells may be stained with one or more antibodies and/or probes prior to encapsulating them into drops.

One or more lysing agents may also be added to the discrete entities (e.g., droplets), containing a cell, under conditions in which the cell(s) may be caused to burst, thereby releasing their genomes. The lysing agents may be added after the cells are encapsulated into discrete entities. Any convenient lysing agent may be employed, such as proteinase K or cytotoxins. In particular embodiments, cells may be co-encapsulated in drops with lysis buffer containing detergents such as Triton X100 and/or proteinase K. The specific conditions in which the cell(s) may be caused to burst will vary depending on the specific lysing agent used. For example, if proteinase K is incorporated as a lysing agent, the discrete entities (e.g., droplets), may be heated to about 37-60° C. for about 20 min to lyse the cells and to allow the proteinase K to digest cellular proteins, after which they may be heated to about 95° C. for about 5-10 min to deactivate the proteinase K. In certain aspects, cell lysis may also, or instead, rely on techniques that do not involve addition of lysing agent. For example, lysis may be achieved by mechanical techniques that may employ various geometric features to effect piercing, shearing, abrading, etc. of cells. Other types of mechanical breakage such as acoustic techniques may also be used. Further, thermal energy can also be used to lyse cells. Any convenient methods of effecting cell lysis may be employed in the methods described herein as appropriate.

One or more primers may be introduced into the discrete entities for each of the genes to be detected. Hence, in certain aspects, primers for all target genes may be present in the discrete entity at the same time, thereby providing a multiplexed assay. The discrete entities may be temperature-cycled so that discrete entities will undergo PCR. In certain embodiments, rolling circle amplification (RCA)-based proximity ligation is employed.

In some embodiments, a surfactant may be used to stabilize the discrete entities. In some cases, the discrete entities or the associated emulsion lack a surfactant. Accordingly, a discrete entity may involve a surfactant stabilized emulsion. Any convenient surfactant that allows for the desired reactions to be performed in the discrete entities, may be used. In other aspects, a discrete entity is not stabilized by surfactants or particles. The surfactant used depends on a number of factors such as the oil and aqueous phases (or other suitable immiscible phases (e.g., any suitable hydrophobic and hydrophilic phases)) used for the emulsions. For example, when using aqueous droplets in a fluorocarbon oil, the surfactant may have a hydrophilic block (PEG-PPO) and a hydrophobic fluorinated block (Krytox® FSH). If, however, the oil was switched to be a hydrocarbon oil, for example, the surfactant would instead be chosen so that it had a hydrophobic hydrocarbon block, like the surfactant ABIL EM90. In selecting a surfactant, desirable properties that may be considered in choosing the surfactant may include one or more of the following: (1) the surfactant has low viscosity; (2) the surfactant is immiscible with the polymer used to construct the device, and thus it doesn't swell the device; (3) biocompatibility; (4) the assay reagents are not soluble in the surfactant; (5) the surfactant exhibits favorable gas solubility, in that it allows gases to come in and out; (6) the surfactant has a boiling point higher than the temperature used for PCR (e.g., 95° C.); (7) the emulsion stability; (8) that the surfactant stabilizes drops of the desired size; (9) that the surfactant is soluble in the carrier phase and not in the droplet phase; (10) that the surfactant has limited fluorescence properties; and (11) that the surfactant remains soluble in the carrier phase over a range of temperatures. Other surfactants can also be envisioned, including ionic surfactants. Other additives can also be included in the oil to stabilize the discrete entities including polymers that increase discrete entity stability at temperatures above 35° C.

The discrete entities (e.g., microdroplets) described herein may be prepared as emulsions, such as an aqueous phase fluid dispersed in an immiscible phase carrier fluid (e.g., a fluorocarbon oil or a hydrocarbon oil) or vice versa. In some cases, the carrier fluid comprises a fluorinated compound. In some cases, the carrier fluid is an aqueous fluid. The nature of the microfluidic channel (or a coating thereon) (e.g., hydrophilic or hydrophobic), may be selected so as to be compatible with the type of emulsion being utilized at a particular point in a microfluidic workflow.

Emulsions may be generated using microfluidic devices. Microfluidic devices can form emulsions composed of droplets that are uniform in size. The microdroplet generation process may be accomplished by pumping two immiscible fluids, such as oil and water, into a junction. The junction shape, fluid properties (viscosity, interfacial tension, etc.), and flow rates influence the properties of the microdroplets generated but, for a relatively wide range of properties, microdroplets of controlled, uniform size can be generated using methods like T-junctions and flow focusing. To vary microdroplet size, the flow rates of the immiscible liquids may be varied since, for T-junction and flow focus methodologies over a certain range of properties, microdroplet size depends on total flow rate and the ratio of the two fluid flow rates. To generate an emulsion with microfluidic methods, the two fluids are normally loaded into two inlet reservoirs (syringes, pressure tubes) and then pressurized as needed to generate the desired flow rates (using syringe pumps, pressure regulators, gravity, etc.). This pumps the fluids through the device at the desired flow rates, thus generating microdroplet of the desired size and rate.

In some cases, a cell in a discrete entity may be labeled (e.g., by a fluorescent label, a barcode, or a combination thereof). In practicing the subject methods, a number of reagents may be incorporated into and/or encapsulated by, the discrete entities in one or more steps (e.g., about 2, about 3, about 4, or about 5 or more steps). Such reagents may include, for example, amplification reagents, such as Polymerase Chain Reaction (PCR) reagents. The methods of adding reagents to the discrete entities may vary in a number of ways. Approaches of interest include, but are not limited to, those described by Ahn, et al., Appl. Phys. Lett. 88, 264105 (2006); Priest, et al., Appl. Phys. Lett. 89, 134101 (2006); Abate, et al., PNAS, Nov. 9, 2010 vol. 107 no. 45 19163-19166; and Song, et al., Anal. Chem., 2006, 78 (14), pp 4839-4849; the disclosures of which are incorporated herein by reference. For instance, a reagent may be added to a discrete entity by a method involving merging a discrete entity with a second discrete entity which contains the reagent(s) in a discrete entity merger region of a microfluidic device described herein.

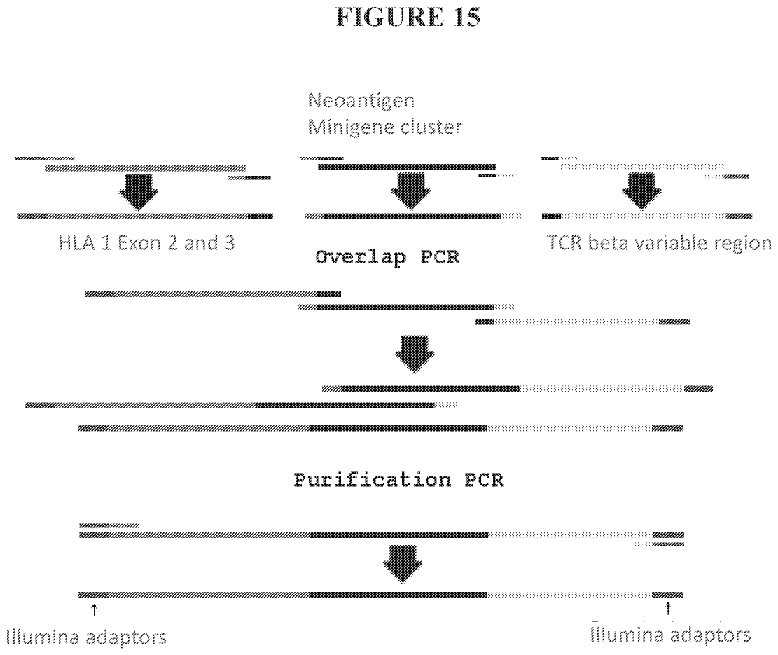

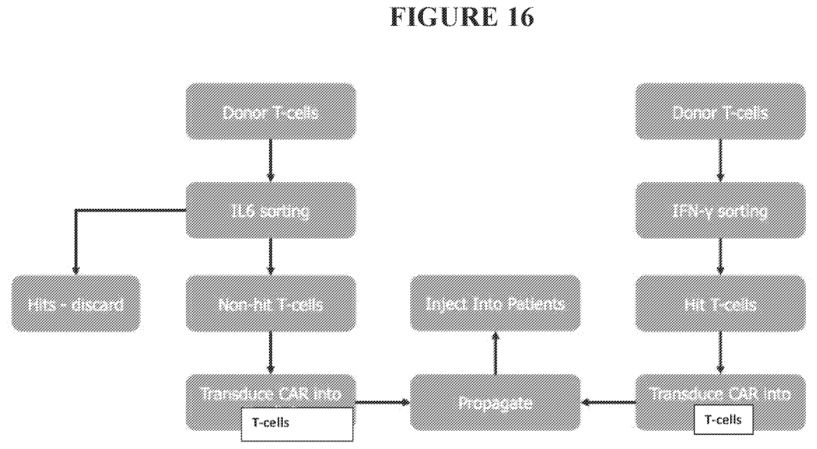

In certain embodiments, overlap extension PCR (OE-PCR) is employed to both amplify nucleic acid from cells (e.g., SD cells and/or T-cells), but also to associate the nucleic acid encoding the neoantigen from the SD cell with the nucleic acid encoding the TCR from the T-cell (See, e.g., FIG. 15). For example, after imaging a droplet and finding SD cells bound to T-cells (e.g., based on cytokine release from the T-cells), such cells could be lysed releasing the nucleic acid. To such droplets, Drop-seq beads and RT-PCR reagents are added to capture the mRNAs on the Drop-seq bead. The PCR could use overlap extension PCR to associate the variable region nucleic acid with the neoantigen nucleic acid. In other embodiments the SD cell could be dendritic cells (DCs) isolated from patient PBMCs. DNA corresponding to minigene clusters would be packaged in lentiviral particles and used to transfect patient DCs. The DCs displaying neoantigens can then be used in a designer droplet assay with isolated patient T-cells and evaluated for cytokine secretion. Assay positive designer droplets can then be used as the starting point for OE-PCR where nucleic acid encoding patient Human Leukocyte Antigen (HLA) type can be associated to nucleic acid encoding neoantigens and the variable region of TCR thereby yielding matched HLA, neoantigen and variable region of TCR sequences. Primers for amplifying HLA I and II regions from cDNAs are previously known in the art (e.g., Hansen et al., BMC Genomics volume 13, Article number: 37 (2012), herein incorporated by reference).). Details on overlap extension PCR are found in the art, including at, for example, U.S. Pat. Pub. 20150154352 and Turchaninova et al., Eur. J. Immunol. 2013. 43: 2507-2515, both of which are herein incorporated by reference. One of the advantages of this method is there will not be a need for barcoding individual assay positive droplets because the nucleic acid encoding the neoantigen and TCR will be linked in one fragment.

One or more reagents may also, or instead, be added using techniques such as droplet coalescence, or picoinjection. In droplet coalescence, a target drop may be flowed alongside a microdroplet containing the reagent(s) to be added to the droplet. The two droplets may be flowed such that they are in contact with each other, but not touching other microdroplets. These drops may then be passed through electrodes or other aspects for applying an electrical field, wherein the electric field may destabilize the microdroplets such that they are merged together. Reagents may also, or instead, be added using picoinjection. In this approach, a target drop may be flowed past a channel containing the reagent(s) to be added, wherein the reagent(s) are at an elevated pressure. Due to the presence of the surfactants, however, in the absence of an electric field, the microdroplet will flow past without being injected, because surfactants coating the microdroplet may prevent the fluid(s) from entering. However, if an electric field is applied to the microdroplet as it passes the injector, fluid containing the reagent(s) will be injected into the microdroplet. The amount of reagent added to the microdroplet may be controlled by several different parameters, such as by adjusting the injection pressure and the velocity of the flowing drops, by switching the electric field on and off, and the like.

In some cases, a discrete entity includes a bead. In some cases, at least one dimension of the bead (e.g., diameter, is between about 0.5 μm and about 500 μm). In some cases, the bead is made of a polymeric material, such as polystyrene. In some cases, the bead is magnetic or contains a magnetic component. In some cases, the bead has a biomolecule attached to its surface, such as an antibody, a protein, an antigen, DNA, RNA, streptavidin, or a combination thereof. In some cases, the bead is an immunoassay bead. In some cases, the bead is an RNA capture bead. As such, the present disclosure provides methods of selectively combining a biomolecule with another compound or cell, wherein the method includes selectively isolating the biomolecule from a composition using the bead, making a discrete entity that includes the bead and biomolecule, and selectively combining the discrete entity containing the bead and biomolecule with one or more other discrete entities that contain one or more other compounds or cells using the microfluidic device described herein. Methods of selectively isolating biomolecules using beads are known in the art, e.g. U.S. 2010/0009383, which is incorporated herein by reference for its disclosure of a method of separating a biomolecule or cell using beads.

In some embodiments, the methods, devices, and/or systems described herein can be used to detect nucleic acids, such as the neoantigen on the surface of the SD cells or the TCR from T-cells. In certain embodiments, reagents necessary for amplification are added to the droplets, either by combining them with the sample droplets prior to dispensing, or by dispensing additional droplets to the positions of the sample containing droplets, wherein the additional droplets include the necessary reagents and a detection component, where the detection component signals the amplification. The droplets are then incubated under conditions suitable for amplification and monitored to read the detection component. This provides, for each droplet, a rate of change of the detection component which can be used to detect and/or quantitate the nucleic acids in the droplets.

In some embodiments, the methods, devices, and/or systems described herein can be used to sequence nucleic acid derived from single cells. For example, individual cells can be encapsulated in the droplets and dispensed to the substrate as described herein. The cells can then be lysed and subjected to molecular biological processing to amplify and/or tag their nucleic acids with barcodes. The material from all the droplets can then be pooled for all cells and sequenced and the barcodes used to sort the sequences according to single droplets or cells. These methods can be used, for example, to sequence the genomes or transcriptomes of single cells in a massively parallel format.

As described above, in certain embodiments, nucleic acid sequence assay components that employ barcoding for labelling individual mRNA molecules, and/or for labeling for cell/well source (e.g., if wells pooled before sequencing analysis), and/or for labeling particular affixed entities (e.g., if droplet from two or more affixed entities are pooled prior to sequencing) are employed. Examples of such barcoding methodologies and reagents are found in Pat. Pub. US2007/0020640, Pat. Pub. 2012/0010091, U.S. Pat. Nos. 8,835,358, 8,481,292, Qiu et al. (Plant. Physiol., 133, 475-481, 2003), Parameswaran et al. (Nucleic Acids Res. 2007 October; 35(19): e130), Craig et al. reference (Nat. Methods, 2008, October, 5(10):887-893), Bontoux et al. (Lab Chip, 2008, 8:443-450), Esumi et al. (Neuro. Res., 2008, 60:439-451), Hug et al., J. Theor., Biol., 2003, 221:615-624), Sutcliffe et al. (PNAS, 97(5):1976-1981; 2000), Hollas and Schuler (Lecture Notes in Computer Science Volume 2812, 2003, pp 55-62), and WO201420127; all of which are herein incorporated by reference in their entireties, including for reaction conditions and reagents related to barcoding and sequencing of nucleic acids.

In certain embodiments, the DropSeq method employing beads with primers attached to them are employed to sequence the noeantigens or TCRs. An example of such a method is described in Macosko et al., Cell, 161(5):1202-1214 (see, e.g., FIG. 1), which is herein incorporated by reference in its entirety. In certain embodiments employing DropSeq, barcoded template switch oligos are bound to beads and oligo dT is supplied in solution along with RT PCR reagents. Reverse transcription (RT) can, for example, be performed as described in Kim et al., Anal Chem. 2018 Jan. 16; 90(2):1273-1279, herein incorporated by reference. In other embodiments, barcoded oligo-dT beads are provided, the cells are lysed, mRNAs is captured on the beads, the emulsion is broken, and the drop is re-emulsified to capture mRNA beads with barcoded TSO beads where the TSO can be released by UV. Solution phase TSO can then be used for performing RT-PCR. Primers specific to the variable regions displayed on the surface of the SD cells can be employed to amplify such variable regions prior to sequencing.

In certain embodiments, unique oligo drops are provided to the fixed entities, and allow a link between imaging and genomics. For example, the unique oligos can contain two part 8 mer barcodes linked to polyA or TSO followed by 8-mer barcodes. In this regard, if one employs 96 barcoded oligos, selecting any three can generate 142,880 combinations. It is known what combination of three oligos are printed at each well position to identify that particular well (e.g., so a neoantigen that binds a TCR and activates the T-cell can be identified). These oligos will also be sequenced and so when one sees a particular 3-oligo combination in the sequencing readouts, one knows the fixed entity and the image for that fixed entity.

In certain embodiments, the barcode tagging and sequencing methods of WO2014201273 (“SCRB-seq” method, herein incorporated by reference) are employed. The necessary reagents for the SCRB-seq method (e.g., modified as necessary for small volumes) are added to the fixed entities, each containing a lysed cells. Briefly, the SCRB-seq method amplifies an initial mRNA sample from cells from a single fixed entity. Initial cDNA synthesis uses a first primer with: i) N6 for cell/well identification, ii) N10 for particular molecule identification, iii) a poly T stretch to bind mRNA, and iv) a region that creates a region where a second template-switching primer will hybridize. The second primer is a template switching primer with a poly G 3′ end, and 5′ end that has iso-bases. After cDNA amplification, the tagged cDNA single fixed entity samples are pooled. Then full-length cDNA synthesis occurs with two different primers, and full-length cDNA is purified. Next, a NEXTERA sequencing library is prepared using an i7 primer (adds one of 12 i7 tags to identify particular multi-well plates) and P5NEXTPT5 to add P5 tag for NEXTERA sequencing (P7 tag added to the other end for NEXTERA). The library is purified on a gel, and then NEXTERA sequencing occurs. As a non-liming example, with twelve 17 plate tags, and 384 cell/well-specific barcodes, this allows total of 4,608 single cell transciptomes to be done at once. This method allows for quantification of mRNA transcripts in single fixed entity.

In other embodiments, the barcode tagging and sequencing methods employ concepts from the Multi-seq method. For example, cells are incubated with anchor and co-anchor lipid modified oligonucleotides (LMO) and encapsulated in droplets. Individual barcodes in droplets can hybridize to exposed regions of the LMOs and these barcodes can be used instead of Drop-seq beads. Anchor-coanchor LMOs remain bound to individual cells at 4° C. but can freely equilibrate between cells in a droplet at 37° C. Thus, a specific LMO-barcode combination in each droplet can be used to link two cells in that droplet that can be tracked after emulsion breaking. In one example, a unique LMO-barcode combination can be randomly assembled in every microfluidic droplet. Barcodes may also be deterministically pre-printed to a microwell array, and additionally provide linkage to imaging data recoded at specific microwell positions. In another embodiment, one cell in each combination may be LMO-barcoded before the combination in droplets. During incubation at 37° C., the LMO-barcodes will re-equilibrate to the initially non-barcoded cell and provide lasting information about co-encapsulation. If a unbarcoded T-cell is combined with an LMO-barcoded antigen presenting cell (APC), this process will allow the type of APC to be read out by sequencing only the T-cell.

In practicing the methods of the present disclosure, one or more sorting steps may be employed. A sorting step sorts a discrete entity into one of two or more locations (e.g. into one of two or more fluid channels). In some cases, the sorting is into one of two fluid channels. Discrete entities are sorted based on one or more properties of the discrete entity or a component within the discrete entity. In addition, such sorting may either be passive sorting or active sorting. Active sorting includes the detection of one or more properties of a discrete entity, or a component within the discrete entity, and sorting based on the detected property. Passive sorting involves sorting a discrete entity without the active detection of a property. Sorting approaches of interest include, by are not necessarily limited to, approaches that involve the use of one or more sorting channels and one or more sorting elements.

Sorting approaches which may be utilized in connection with the disclosed methods, systems and devices also include those described herein, and those described by Agresti, et al., PNAS vol. 107, no 9, 4004-4009. For active sorting, the device includes one or more sorting elements and one or more detectors, wherein each detector is configured to detect one or more properties of a discrete entity, or a component within the discrete entity, and each sorting element is configured to sort the discrete entity into one of two or more locations based on the detecting by the detection element. In some cases, a sorting element is positioned in proximity to the sorting channel, such as an electrode in proximity to the sorting channel. In some cases, a sorting element is positioned within the sorting channel, such as a partial height flow divider in a sorting channel. In some cases, the device includes a sorting element positioned within the sorting channel and one or more sorting elements positioned in proximity to the sorting channel. Exemplary structures and methods for active sorting discrete entities are described in Cole et al., PNAS, 2017, 114, 33, 8728-8733; Clark et al., Lab Chip, 2018, 5, 18, 710-713; and Sciambi et al., Lab on a Chip, 2015, 15, 47-51, the disclosures of which are incorporated herein by reference for sorting elements.

In some cases, the sorting element comprises an electrode configured to exert a dielectrophoretic force, an electrode configured to exert an electrophoretic force, an element configured to exert an acoustic force, a valve, or a combination thereof. In some cases, a sorting element comprises an electrode that is positioned in proximity to the sorting channel, e.g. an electrode configured to exert a dielectrophoretic force on the discrete entity or an electrophoretic force on the discrete entity. In some cases, the electrode is configured to exert an electrophoretic force on the discrete entity. The dielectrophoretic force on the discrete entity can be directed towards the electrode. In some cases, the sorting electrode is a liquid electrode, such as a microfluidic channel containing a conductive material, such as salt water, liquid metal, molten solder, or a conductive ink to be annealed later. In some cases, the electrodes are micropatterned onto a planar surface and the microfluidic device is bonded to the surface. In some case, the electrodes are patterned on the substrate of the microfluidic device, e.g. a patterned indium tin oxide (ITO) glass slide. In some cases, the sorting element includes a selectively actuatable bipolar sorting electrode. In some cases, the sorting element includes two electrodes. In some cases, the sorting element includes a selectively actuatable bipolar droplet sorting electrode. In some cases, the electrode is a solid electrode prepared from any suitable conductive material may be utilized.

In some cases, the sorting element includes two sorting electrodes. In some cases, the two sorting electrodes have substantially different shapes, such as shown in FIG. 2. In some cases, the two sorting electrodes produce electric field lines with substantially different shapes. In some cases, the shapes are such that the pair of electrodes provide a constant electric field gradient. As such, a discrete entity can be subjected to the sorting force for a longer period of time and over a longer distance, thereby allowing a lower voltage to be used. In some cases, the electric field points radially inwards. In some cases, a portion of a first sorting electrode is positioned in the center of the arc of a concentric or essentially concentric sorting channel, and the second sorting electrode is positioned on a side of the sorting channel opposite the first sorting electrode, such as shown in FIG. 2. In some cases, the sorting channel defines a concentric or approximately concentric flow path, wherein a portion of a sorting electrode is located at the center of the concentric or approximately concentric flow path. In some cases, two sorting electrodes are positioned on the same side of the sorting channel. In such embodiments, the shortest distance between the two sorting electrodes is between about 20 μm and about 500 μm, such as between about 50 μm and about 200 μm, between about 75 μm and about 150 μm, between about 100 μm and about 150 μm, or between about 120 μm and about 140 μm. In some cases, the shortest distance between a sorting electrode and the interior of the sorting channel is between about 5 μm and about 100 μm, such as between about 10 μm and about 50 μm, between about 20 μm and about 40 μm, between about 25 μm and about 35 μm, or between about 28 μm and about 32 μm.

In some embodiments, the present disclosure provides microfluidic devices with an improved sorting architecture, which facilitates the high-speed sorting of discrete entities, e.g., microdroplets. This sorting architecture may be used in connection with other embodiments described herein or in any other suitable application where high-speed sorting of microdroplets is desired. Related methods and systems are also described. For example, in some embodiments, a microfluidic device may include a sorting channel; a first outlet channel in fluid communication with the sorting channel; a second outlet channel in fluid communication with the sorting channel; and a dividing wall separating the first outlet channel from the second outlet channel, wherein the dividing wall comprises a first proximal portion having a height which is less than the height of the inlet channel and a second distal portion having a height which is equal to or greater than the height of the inlet channel.

In some cases, the discrete entity is detected while the discrete entity is in the inlet channel via an optical property. In some cases, the optical property is fluorescence. Thus, in some cases, the detector includes an excitation light source and a fluorescence detector. In some cases, the excitation light includes visible light, ultraviolet light, or a combination thereof. In some cases, the detector is an optical scanner. In some cases, the detector includes optical fibers for directing excitation light onto the discrete entity, for directing fluorescent light to a fluorescence detector, or a combination thereof. In some cases, a suitable optical scanner utilizes a laser light.

A variety of different components can be included in the discrete entities to facilitate detection, including one or more fluorescent dyes. Such fluorescent dyes may be divided into families, such as fluorescein and its derivatives; rhodamine and its derivatives; cyanine and its derivatives; coumarin and its derivatives; Cascade Blue and its derivatives; Lucifer Yellow and its derivatives; BODIPY and its derivatives; and the like. Exemplary fluorophores include indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX), LIZ, VIC, NED, PET, SYBR, PicoGreen, RiboGreen, and the like. Descriptions of fluorophores and their use, can be found in, among other places, R. Haugland, Handbook of Fluorescent Probes and Research Products, 9th ed. (2002), Molecular Probes, Eugene, Oreg.; M. Schena, Microarray Analysis (2003), John Wiley & Sons, Hoboken, N.J.; Synthetic Medicinal Chemistry 2003/2004 Catalog, Berry and Associates, Ann Arbor, Mich.; G. Hermanson, Bioconjugate Techniques, Academic Press (1996); and Glen Research 2002 Catalog, Sterling, Va.

In some embodiments, the microfluidic devices herein include directing the discrete entity to a discrete entity merger region. Accordingly, a device as described herein can include a discrete entity merger region and a trapping element positioned in proximity to the discrete entity merger region. The trapping element can to trap a plurality of discrete entities in the discrete entity merger region for a time sufficient for the plurality of discrete entities to combine to form a combined discrete entity by exerting an electromagnetic force, exerting a mechanical force, applying heat, applying light, exerting an electrical force, providing a reagent, or a combination thereof sufficient. In some cases, the electromagnetic force is a dielectrophoretic force. In some cases, the electromagnetic force is an electrophoretic force. In some cases, the discrete entity merger region includes a feature selected from: a geometric change in a dimension of the first outlet channel, a flow obstacle, a flow divider, a laminating fluid inlet, a valve, or a combination thereof. In some cases, the geometric change is a change in the cross-sectional area of the first outlet channel (e.g., the discrete entity merger region has a larger cross-sectional area than the upstream region). In some cases, the geometric change is a change in one dimension of the first outlet channel (e.g., the discrete entity merger region is narrower than the downstream region). In some cases, the geometric change includes a recess in a channel wall. In some cases, the recess includes an area that is not colinear with the flow of fluid from the upstream region, such as shown as item 107 in FIG. 2. In some cases, where a valve is utilized, the valve is configured to switch between at least two states. In some cases, in the first state, the valve impedes the flow of a discrete entity past the discrete entity merger region while allowing flow of the carrier fluid past the discrete entity merger region. In some cases, in the second state, the valve is configured such that the combined discrete entity is not impeded from flowing past the discrete entity region. In some cases, the method includes putting the valve in a first state such that discrete entities can be trapped and combined into a combined discrete entity, and then putting the valve into a second state to release the discrete entity from the discrete entity merger region. In some cases, the valve is a membrane valve.

A laminating fluid inlet functions in a similar manner to certain embodiments of the spacer fluid inlet described above, such as a laminating fluid inlet is configured such that flowing fluid through the laminating fluid inlet will cause a discrete entity to move further away from a first side a channel and closer to a second side of a channel. Stated in another manner, the fluid flowing through the laminating fluid inlet contacts the fluid moving into the discrete entity merger region from an upstream region of the first outlet channel, thereby affecting the flow of fluid coming from the upstream region. In some cases, the fluid is oil, or a fluid which is otherwise immiscible with the fluid of the discrete entity.

FIG. 2 shows an embodiment wherein the discrete entity merger region includes recess 107, flow divider 113, and laminating fluid inlet 112. In FIG. 2, the laminating fluid provides a force pushing a discrete entity into recess 107 and towards trapping electrodes 109. In addition, flow divider 113 in FIG. 2 further affects the interaction of the laminating fluid and the fluid coming from the upstream region, thereby increasing the force pushing the discrete entity into recess 107. As such, a discrete entity merger region according to the present disclosure can include a laminating oil inlet and/or a flow divider, wherein such an element or elements are configured such that flowing oil through the laminating oil inlet channel will produce a force pushing a discrete entity in the discrete entity merger region towards a trapping electrode, a recess, or a combination thereof. In some embodiments, the device can include a flow divider without the laminating fluid inlet.

In some cases, the downstream region of the first outlet channel is configured to aid in the trapping of a discrete entity in the discrete entity merger region. In some cases, the downstream region has a larger cross-sectional area than the discrete entity merger region, which is an example of a geometric change in the first outlet channel. In some cases, the downstream region has a triangular or approximately triangular shape. In some cases, the downstream region has a triangular or approximately triangular shape and the discrete entity merger region is located at or near a vertex of the triangle. As an example, in the system of FIG. 3 has downstream region 208 and discrete entity merger region 207. In some cases, the longitudinal axis of the downstream region is parallel to the longitudinal axis of the discrete entity merger region, whereas in other cases such longitudinal axes are not parallel. In some cases, such axes are parallel but not colinear. In some cases, the axes are parallel and colinear. In some cases, the angle between such axes is greater than 0°, such as 5° or more, 10° or more, 15° or more, 30° or more, 45° or more, 60° or more, 75° or more, 90° or more, 135° or more, or 175° or more. In some cases, such an angle is between approximately 15° and approximately 135°. In some cases, such an angle is between approximately 600 and approximately 120°, such as shown in FIG. 3.

In some embodiments, the trapping element includes one or more electrodes, such as an electrode configured to exert a dielectrophoretic force on the discrete entity. In some cases, the electrode is configured to exert an electrophoretic force. The dielectrophoretic force on the discrete entity can be directed towards the electrode (i.e. an attractive force), away from the electrode (i.e. a repulsive force), or in any other direction. In some cases, the trapping electrode is a liquid electrode, such as a microfluidic channel containing a conductive material, e.g. salt water, liquid metal, molten solder, or a conductive ink to be annealed later. In some case, the electrodes are patterned on the substrate of the microfluidic device, e.g. a patterned indium tin oxide (ITO) glass slide. In some cases, the trapping element includes a selectively actuatable bipolar trapping electrode. In some cases, the trapping element includes two electrodes. In some cases, is the trapping element includes a selectively actuatable bipolar droplet trapping electrode. In some cases, the electrode is a solid electrode prepared from any suitable conductive material may be utilized. In some cases, the trapping element includes three or more trapping electrodes, such as four or more, five or more, ten or more, or twenty or more. In such cases, the trapping electrodes can be configured to form two or more bipolar electrode pairs, such as three or more pairs, four or more pairs, five or more pairs, or ten or more pairs.

In some cases, the sorting element sorts discrete entities at a rate of at least 10 Hz, such as at least 100 Hz, at least 500 Hz, at least 1,000 Hz, at least 2,000 Hz, or at least 10,000 Hz. In some cases, only 50% or less of the discrete entities contain the contents desired for the second discrete entity, such as 25% or less, 10% or less, 5% or less, 1% or less, or 0.1% or less. In some cases, the discrete entity merger region and trapping element are configured to trap a first discrete entity for 0.1 ms or more, such as 1 ms or more, 5 ms or more, 10 ms or more, 25 ms or more, 50 ms or more, 100 ms or more, 500 ms or more, 1,000 ms or more, or 5,000 ms or more. In some cases, a first discrete entity is trapped in the discrete entity merger region for 0.1 ms or more before a second discrete entity enters the region, such as 1 ms or more, 10 ms or more, 100 ms or more, or 1,000 ms or more.

The present disclosure provides a method of selectively performing reactions by selectively combining two or more discrete entities, as described above, wherein the reaction occurs between one or more components from each discrete entity. Such components can be one or more cells, one or more products derived from a cell, one or more reagents, or a combination thereof. In some cases, a suitable method includes combination of one cell and one or more reagents. As an example, FIG. 4 shows the combination of four discrete entities, wherein three of the discrete entities each contain a different reagent and the fourth discrete entity contains a single cell. As such, FIG. 4 shows that a microfluidic device as described herein can be used to selectively combine different discrete entities, resulting in the formation of a combined discrete entity, e.g., that contains the three reagents and the cell. In some cases, the reagents can include cell lysing reagents, PCR reagents, reagents for analyzing the DNA or RNA within a cell, antibodies, or a combination thereof. In such cases, the method can further include collecting genomic data from contents of the discrete entities or combined discrete entities. In some cases, the one or more products derived from a cell include cell lysate, DNA, RNA, or a combination thereof. As such, the method can involve analyzing products from a cell, e.g. cell lysate, even though the cell per se is included in any of the discrete entities.

The present disclosure provides methods of selectively combining two or more discrete entities wherein each discrete entity contains one or more cell (e.g., a T-cell and a cell presenting a neoantigen). In some cases, the ratio of a first type of cell to a second type of cell is 1.1:1.0 or more, e.g. 2:1 or more, 5:1 or more, 10:1 or more, 25:1 or more. The number of cells can be 2:1, 2:1 or more, 5:1 or more, 10:1 or more, 25:1 or more. In other cases, three or more types of cells are combined in unequal ratios or numbers. The ratio or number of each pair of cells can be those numbers and ratios recited above.

In certain embodiments, gene signatures can be used to evaluate T-cells and NK cells (e.g., Chimeric Antigen Receptor-T cells' (CAR-T)) ability to secrete cytokines and identify T-cell specific, or NK-specific, signatures. Algorithms can be used to compute polyfunctionality and immune cell identity.

Polyfunctionality of a T-cell is defined as the T-cell's ability to secrete >2 cytokines/chemokines in response to specific T-cell perturbation. Polyfunctionality of T-cells in general and CAR-T cells in particular have been shown to be correlated to successful treatment outcomes depending on the type of cytokines they secrete. Cytokines have been categorized into effector, stimulatory, chemo-attractive, regulatory and inflammatory based on the impact they have on a patient. For example, T-cells that secrete high amounts of inflammatory cytokines can cause severe side effects whereas T-cells that secrete high amounts of effector cytokines are effective in killing its intended target.

Recent work from academia and industry have focused on evaluating secreted cytokines at a single-cell level using technologies such as single-cell barcoded chips (SCBC). SCBCs use single-cell ELISA methods to evaluate 30+ cytokines from 2000 single-cells and compute a polyfunctional score based on the different categories of cytokines. The polyfunctional scores are then used to identify responders and non-responders of CAR-T therapy.

Computing polyfunctional scores using protein measurements while robust also limits the subset of cytokines that one can interrogate to identify patient responders. An alternative approach is to use gene-expression signatures of single-cells. Single-cell RNA-seq methods have the potential to identify hundreds to thousands of differentially expressed genes. The subset of genes that can be used to evaluate polyfunctionality include genes that encode cytokines, transcription factors and other proteins such as annexin A1 that play a role in the regulation of T-cell activation.

Isoplexis uses single-cell cytokine measurements using ELISA, and therefore relies on secreted protein measurements. The methods described herein use gene expression measurements of single cells that have been sorted based on cytokine measurements. Such methods therefore takes into consideration both secreted and intracellular proteins to compute polyfunctionality.

In certain embodiments, patient T-cells (e.g., regular and CAR-T T-cells) and cultured Raji cells are encapsulated as single-cells and merged to create an assembled droplet that contains a single RAJI cell, a single T-cell and interferon-gamma (or other cytokine) assay reagents. The assembled droplets are incubated, for example, at 37 C for 4-12 hours and the droplets sorted for interferon-gamma signal or other cytokine signal. The droplets are de-emulsified, cell viability measured and subjected to VDJ sequencing and single-cell RNA-seq using 10× Genomics Chromium Next-GEM system. The barcoded cDNAs are further processed according to published instructions from 10× genomics and subjected to DNA sequencing using a NextSeq system. Raw data from the sequencer is analyzed using 10× CLoupe and VLoupe software packages to identify T-cell subtypes based on differences in interferon-gamma signal. The VDJ sequences were superimposed on the scRNA-seq data to identify individual T-cells that showed productive VDJ sequences and RNA-seq data. The top 100 differentially expressed genes in each cluster are identified and each gene's function is evaluated on UniProt to determine the gene's role in T-cell activation, stability, apoptotic response and cytokine response. The log2Fold Change of each individual gene that had a beneficial effect on T-cell health were added together and subtracted from log2Fold Change of genes with adverse effects. For example, LGALS1 encodes galectin and is a strong inducer of T-cell apoptosis. The following genes can be used to compute an expression signature with genes marked in green and red colors (LGALS1) are favorable and adverse to T-cell health.

| T-cell | T-cell |
|---|---|
| polyfunctionality | Identity |
| ANXA1 | CCND2 |
| CCL1 | CD2 |
| CCL3 | CD28 |
| CCL4 | CD247 |
| CCL5 | CD3D |
| CD40LG | CD3E |
| CSF2 | CD3G |
| GZMA | CD44 |
| GZMB | CD7 |
| ICOS | CD96 |
| IFNG | TRAC |
| IL2 | TRAV29DV5 |
| IL2RA | TRBC1 |
| IL13 | TRBV12-3 |
| IL32 | TRBV20-1 |
| LCK | TRBV5-1 |
| LGALS1 | TRBV7-6 |
| TNFRSF9 | |
| TNFRSF18 | |
| TNFRSF4 | |
| TNFRSF14 | |

In certain embodiments, the methods herein use microfluidics (e.g., as described elsewhere herein) to bring together patient T-cells (e.g., CAR-T cells), target cells and commercially available cytokine assay reagents. Such methods allow the ability to link functional analysis to single-cell genomics and VDJ sequencing.

### EXAMPLES

### Example 1

### Sequencing of TCR of Single T-Cell

### TCR Sequencing of Single Jurkat T-Cells

### Single Cell Sorting

Jurkat cells (immortalized T-cell cell line) were cultured in RPMI medium with 10% FBS and antibiotics. Cells were pelleted and washed with PBS two times followed by staining with Cell Tracker DeepRed (Thermo Fisher). Stained cells were counted and encapsulated into droplet using a standard 80 um coflow device at a concentration ˜3×10\hat{ }6 cells/ml. Droplets were injected into 80 um assembler device and single DeepRed positive droplets were sorted into single PCR tubes containing 8 ul of lysis mix (3.75 ul 0.2% TritonX100, 0.25 ul RNAase inhibitor, 2 ul 10 mM dNTP, 2 ul 10 uM oligo dT primer (LL15)).

### Reverse Transcription

3 ul of 1H,1H,2H,2H-Perfluoro-1-octanol (Sigma Aldrich) was added to each tube followed by vortexing and centrifugation to separate oil from the lysis phase. 6 ul of lysis phase was then pipetted out followed by adding 14 ul of reverse transcription reagent based on smart-seq2 protocol (Picelli et al. 2014 Nature Protocol). After reverse transcription at 42 C for 90 mins followed by 9 cycles of 50 C for 2 min and 42 C for 2 min and 70 C for 15 min (1 ul Maxima H Minus reverse transcriptase, 0.5 ul RNAse inhibitor, 4 ul Maxima RT buffer (5×), 1 ul DTT (100 mM), 4 ul Betaine (5 M), 0.12 ul MgCl2 (1 M), 0.2 ul TSO (100 pM) and 3.18 ul Nuclease-free water), 30 ul PCR reagent (25 ul of KAPA hifi 2× master mix, 4 ul 10 uM TSO primer and 1 ul H₂O) was added to the RT reaction followed by cDNA PCR with LL14 (3 min at 98 C and 30 cycles of 98 C 20 s, 67 C 15 s, 72 C 3 min and 72 C for 5 min). cDNA PCR was purified using Ampure beads (0.6×) followed by bioanalyzer to check the quality of the amplified cDNA.

### TCR Amplification

1 ul of purified cDNA PCR was used as template for TCR PCR1 using outer TCRa, TCRb C region primer and TSOshort primer (LL16 with LL6 and LL7) with a TCRa and b primer ratio of 2:1. KAPA HIFI hotstart master mix was used for the PCR. PCR was performed with 98 C 3 min, 16 cycles of 98 C 20 s, 58 C 15 s, 72 C 60 s and 72 C 5 min. 1 ul of PCR products from TCR PCR1 was used as template for TCR PCR2 with LL12 and LL9/LL10 (inner TCRa, TCRb C region primer and TSO primer). The process of TCR PCR1 was then repeated.

### Sequencing Library Prep and Sequencing

For each sample, 1 ul from TCR PCR2 was used with Nextera Index primer N7XX and N5XX for PCR to construct Illumina sequencing compatible library using KAPA hotstart HIFI following PCR protocol (98 C 3 min, 14 cycles of 98 C 20 s, 58 C 15 s, 72 C 45 s and 72 C 5 min). The PCR product (50 ul) was Ampure purified with first 22.5 ul Ampure beads to 50 ul PCR followed by adding 10 ul Ampure beads to ˜70 ul supernatant and was then eluted to 20 ul with H₂O.

Eluted TCR libraries were measured with Qubit high sensitivity DNA assay and quality checked with Bioanalyzer DNA high sensitivity chip and libraries were mixed with equal molarity and sequenced with MiSeq micro kit (PE 300 or 500) with read1 25 bp (read UMI) and read2 275 bp (read TCR).

Read2 from different samples were fed into MiXCR analysis software to extract sequence of TCR a and b from each sample.

### Preparation of IFNγ Beads

5 μm diameter carboxyl-coated polystyrene beads (Spherotech) were functionalized with IFNγ capture antibody following standard EDC carbodiimide chemistry. Briefly, beads were pelleted via centrifugation and resuspended in MES buffer with EDC (2 mM, Sigma Aldrich) and Sulfo-NHS (5 mM, Thermo Fisher) to activate the surface carboxyl groups on the beads. The beads were then washed twice via centrifugation and resuspended in a suspension containing IFNγ capture antibody (50 μg, R&D) and allowed to react overnight at room temperature under constant agitation (final reaction volume of 200 μL). The reaction was then quenched with Tris-HCl and washed twice into PBS. Beads were prepared within one month of use for CAR-T cell activation testing.

### TCR Sequencing of Single Activated CART Cells

CAR-T activation was assessed via coencapsulation of CAR-T cells with Raji cells (antigen presenting cells for CAR-T) and cytokine detection reagents. All three final droplet components were fluorescently labelled and pre-encapsulated in input droplets of 40 μm diameter with FITC (1 μM-5 μM) as a droplet detection dye. CAR-T cells were transduced to constitutively express mCherry which provided a sorting fluorescent signature for these cells. Raji cells were stained with Cell Tracker Violet for sorting purposes. CAR-T cells and Raji cells were encapsulated at limiting dilution to achieve single cell occupancy per individual droplet. Cytokine detection reagents consisted of IFNγ capture beads (prepared as described), IFNγ detection antibody (15 nM), and streptavidin Alexa Fluor 647 (15 nM). These three input drop types were then merged together via the MOD platform and the resulting emulsion was collected and incubated for 8 hours. The assembled drops were then run through a droplet sorting device and droplets displaying a positive signal on the detection bead were sorted and enriched for downstream TCR sequencing following the protocol described for Jurkat cells above.

Primer sequences
S_LL6_TCRa_outer
(SEQ ID NO: 1)
CAGACAGACTTGTCACTGGATTTAG
S_LL7_TCRb_outer
(SEQ ID NO: 2)
CTTTTGGGTGTGGGAGATCTCTG
S_LL9_TCR_alphaNX
(SEQ ID NO: 3)
TGTCTCGTGGGCTCGGAGATGTGTATAAGAGACAGNNGTCACTGGAT
TTAGAGTCTCTCAG
S_LL10_TCR_beta
(SEQ ID NO: 4)
NXTGTCTCGTGGGCTCGGAGATGTGTATAAGAGACAGNNGAGATCTCTGC
TTCTGATGGCTC
S_LL12_TSONXTshort
(SEQ ID NO: 5)
TCGTCGGCAGCGTCAGATGTGTATAAGAGACAGNNTATCAACGCAGAGTG
AATGGG
S_LL16_TSOshort
(SEQ ID NO: 6)
TATCAACGCAGAGTGAATGGG
TSO
(SEQ ID NO: 7)
AAGCAGTGGTATCAACGCAGAGTGAATrGrGrG
S_LL14_TSOpcr
(SEQ ID NO: 8)
AAGCAGTGGTATCAACGCAGAGT
S_LL15_TSOoligodT
(SEQ ID NO: 9)
AAGCAGTGGTATCAACGCAGAGTACNNNNNNNNTTTTTTTTTTT
TTTTTTTTTTTTTTTTTTTVN

### Example 2

T cells from healthy donor R42598 were activated with Human T-Activator CD3/CD28 beads (Dynal) for 24 hours. A 2nd generation lentivirus containing the α-CD19 CAR construct was added to the cells and incubated for no more than 24 hours. T-cells/beads were separated from lentiviral particles via centrifugation and cultured for an additional 2-3 days prior to removing the beads. Transduction efficiency was measured by flow cytometry using mCherry coexpression with the CAR on day 4 post transduction. Measured transduction efficiency was 42% for donor R42446 and 43% for donor R42598. The Human T-Activator CD3/CD28 beads were removed on day 7 for patient R42598 and the T cells were expanded in Lonza's X-Vivo 15 supplemented with 5% Human AB Serum, 1 mM N-Acetyl Cysteine, 55 uM Beta Mercaptoethanol, and 2 mM GlutaMAX until all of the cells transitioned to a resting state at day 11-15 post transduction. A) Over 20000 droplets were assembled in a 1:1 ratio to contain a single patient CAR-T cell and a target RAJI cell and merged with IFN-γ assay reagents in droplets. Droplets were incubated for 12 hours at 37° C. in an incubator (5% CO2). Assayed droplets were sorted for IFN-γ signal, and ˜2000 droplets were collected for the sorted AND waste

IFN-γ+ve droplets were sorted using the microfluidic device described herein and droplets that were sorted as well as collected in waste were separately de-emulsified, single-cells retrieved, and viability measured before subjecting the cells to scRNA-seq/TCR-seq using the 10× Genomics NextGEM system. The processed single cells were sequenced using NextSeq 500 to an average sequencing depth of 25-50 k reads/cell and average genes/cell of 1 k-2 k. ScRNA-seq reads were aligned to GRCh38 reference genome and quantified using Cell Ranger (10× Genomics, version 3.1.0). Filtered gene-barcodes containing only barcodes with UMI counts passing threshold for cell detection set by Cell Ranger were used for further analysis. Using the Loupe Cell Browser, UMAP projections were derived from 5-6 groups of clusters identified by K-means clustering and the elbow point for determining K was set by excluding clusters with <5 cell-barcodes.

Polyfunctional Gene Expression Scores (PGES) and T-cell Gene Expression Scores (TGES): Top 100 differentially expressed significant genes from Sort and Waste were evaluated for each cluster for polyfunctionality or T-cell specific signatures. Each gene's role in T-cell function (cytokine secretion) or T-cell identity was manually evaluated. Twenty genes that promote or inhibit T-cell function and 17 genes for T-cell identity were used as signatures.

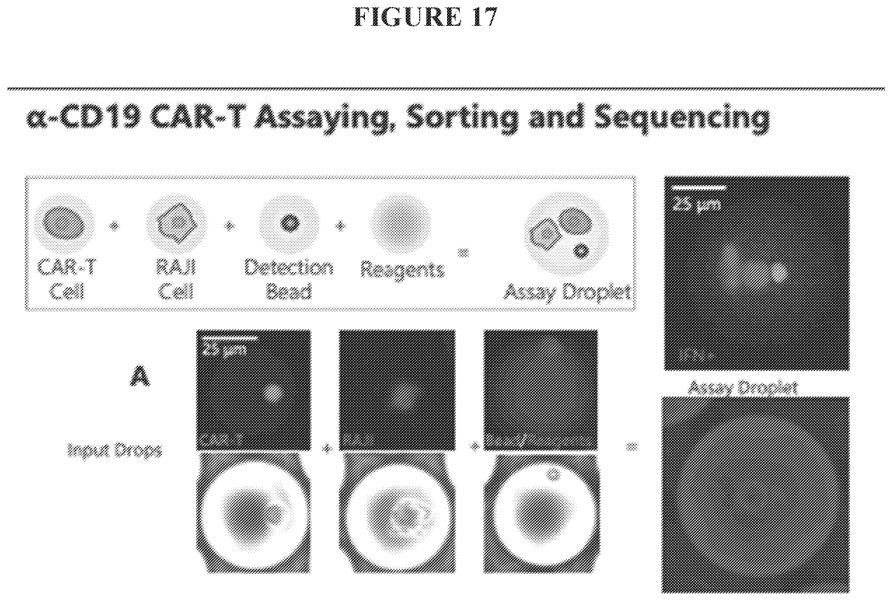

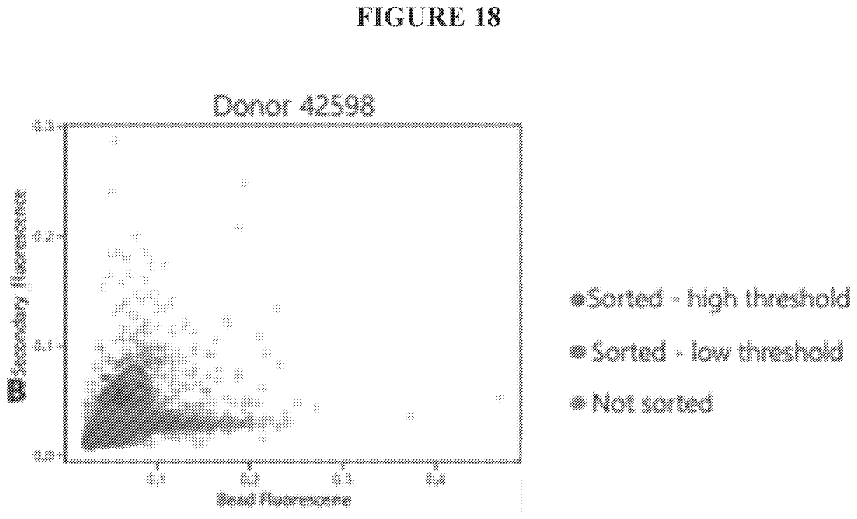

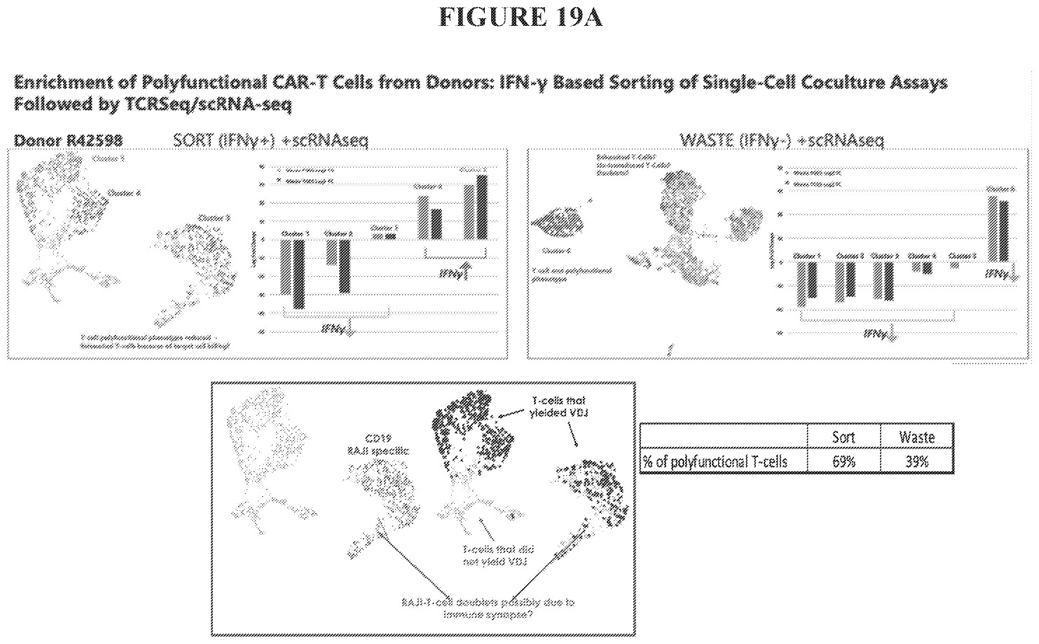

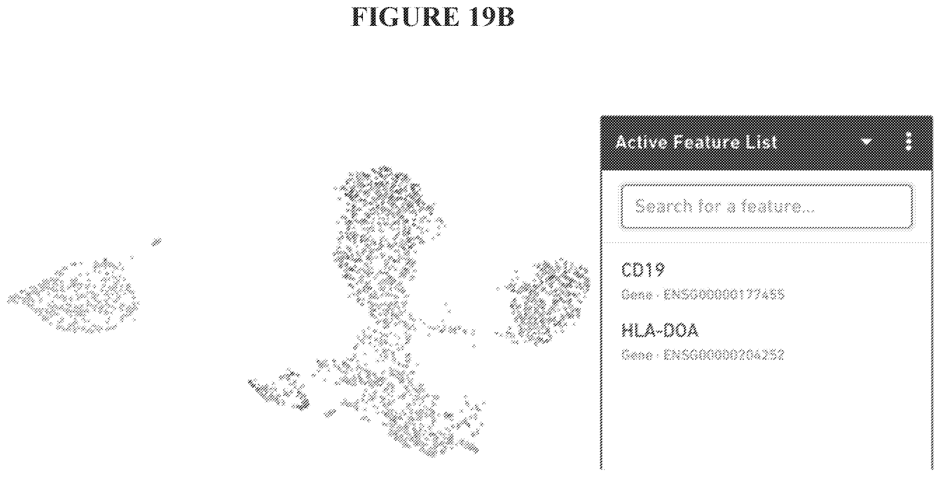

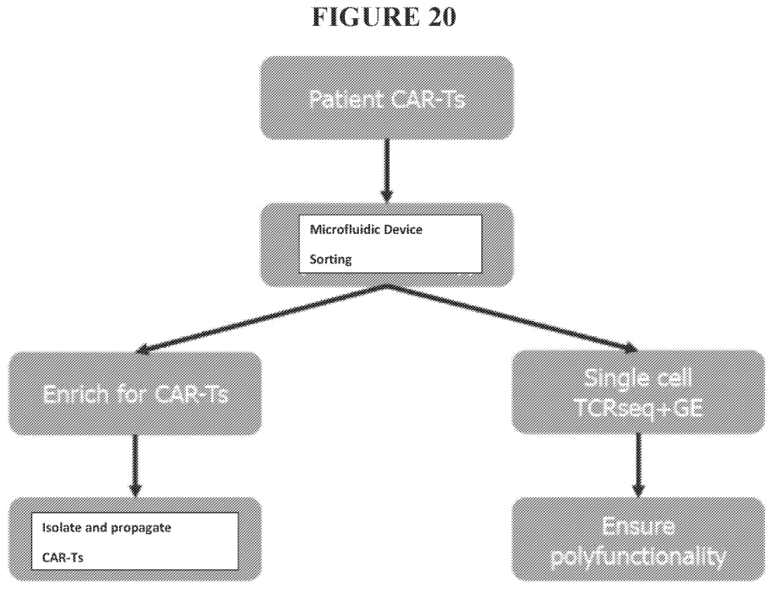

Results are shown in FIGS. 17, 18, and 19. The data suggests the following:
1. Functional sorting of T-cells based on IFNγ signal yields high level granularity of T-cell populations
   Sorted population contains T-cell clusters whose transcriptional profiles exhibit high and low T-cell polyfunctionality
   Many barcodes are shared between T-cells and RAJI cells in cluster 3, which suggests CAR-T-cell mediated killing and T-cell exhaustion
   Low T-cell polyfunctionality may result from IFNγ false-positives from low sorting gate thresholds
   Waste population contains one T-cell cluster with high polyfunctional scores but 1FN-γ is not one of the top 100 upregulated genes.
2. Single RAJI-T-cell cocultures identify putative doublet cell populations that may be indicative of immune synapses.

| T-cell Polyfunctionality | T-cell identity |
|---|---|
| ANXA1 | CCND2 |
| CCL1 | CD2 |
| CCL3 | CD28 |
| CCL4 | CD247 |
| CCL5 | CD3D |
| CD40LG | CD3E |
| CSF2 | CD3G |
| GZMA | CD44 |
| GZMB | CD7 |
| ICOS | CD96 |
| IFNG | TRAC |
| IL2RA | TRAV |
| IL13 | TRBV |
| IL32 | |
| LCK | |
| LGALS1 | |
| TNFRSF9 | |
| TNFRSF18 | |
| TNFRSF4 | |
| TNFRSF14 | |
| SRGN | |

PGES=Σ Log2 Fold Changes(Promotor genes)−Σ Log2 Fold Changes(Inhibitor genes). TGES=Σ Log2 Fold Changes (Identity genes).

### Example 3

### Spheroid Assembly

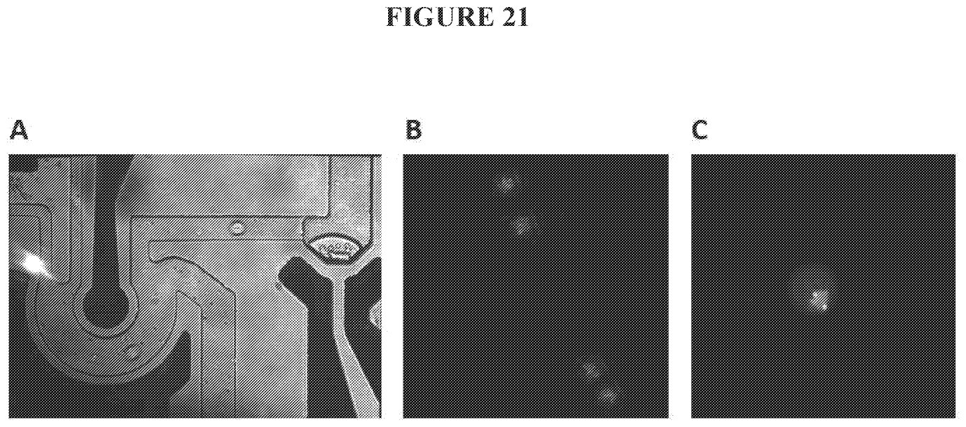

HEPG2 cells (human liver cancer cell line) were cultured in RPMI medium with 10% FBS and antibiotics. Cells were pelleted and washed with PBS two times followed by staining with Cell Tracker DeepRed (Thermo Fisher). Stained cells were counted and encapsulated into droplets using a standard 80 um flow focusing droplet maker at a concentration ˜3×10\hat{ }6 cells/ml. Droplets were reinjected into a 40 um assembler device with a 150 um tall channel in proximity to the assembly trap. This additional height provides a larger available volume in the assembly region and therefore room for more total droplets to be combined (FIG. 21A). This device was used to assemble 20 droplets containing single HEPG2 cells into a merged droplet, which was repeated to provide 2000 duplicated assemblies. The assembled droplets were collected into a 1.5 mL microcentrifuge tube containing 100 uL of dummy emulsion, and incubated offline for 16 hrs. Images of the droplets immediately after assembly showed individual cells (FIG. 21B), but after 16 hours of incubation, the cells had condensed to form a compact spheroid (FIG. 21C).

### Example 4

### Single Droplet Sorting Using a Coalescence Sorter

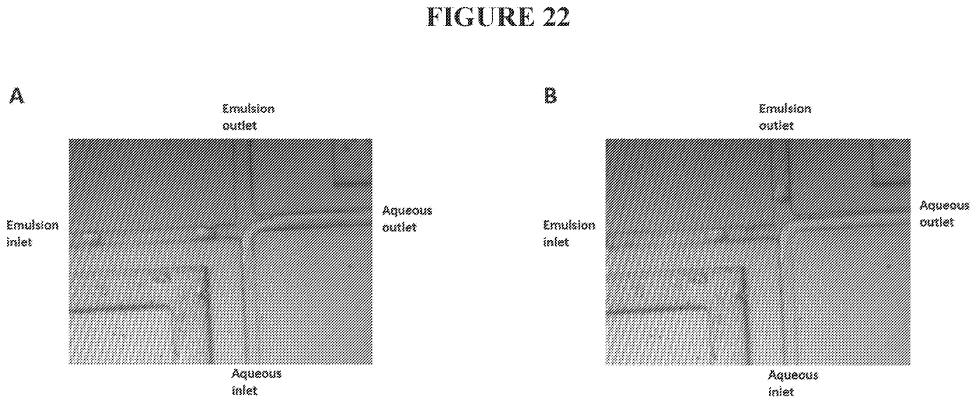

A 50 um tall selective coalescence device was constructed using standard soft lithography techniques. The aqueous and emulsion inlets both have widths of 40 um and the outlets each have widths of 60 um. Upstream of the emulsion inlet, aqueous in oil droplets are made using T-junction drop-maker geometry, with the droplets containing 1 uM fluorescein for detection. The droplet aqueous flow rate is 25 ul/hr and the emulsion oil (HFE 7500 and containing 1% fluorosurfactant) flows at 2000 ul/hr. The coalescence aqueous flow contains 1M NaCl and flows at 4000 ul/hr. Before droplets reach the junction of the emulsion/aqueous junction, they are detected fluorescently. An electrical signal of 700V at 10 kHz is constantly applied to the aqueous stream so that all droplets from the emulsion merge into the aqueous stream and exit through the aqueous outlet (FIG. 22A). When a desired droplet is detected, the electrical signal to the aqueous stream is temporarily turned off, allowing the droplet to bypass the aqueous stream and travel in the emulsion stream to microfluidic architecture downstream.

### Example 5

### Combinatorial Assays Using Expanded Clonal Colonies of Immune and Target Cells

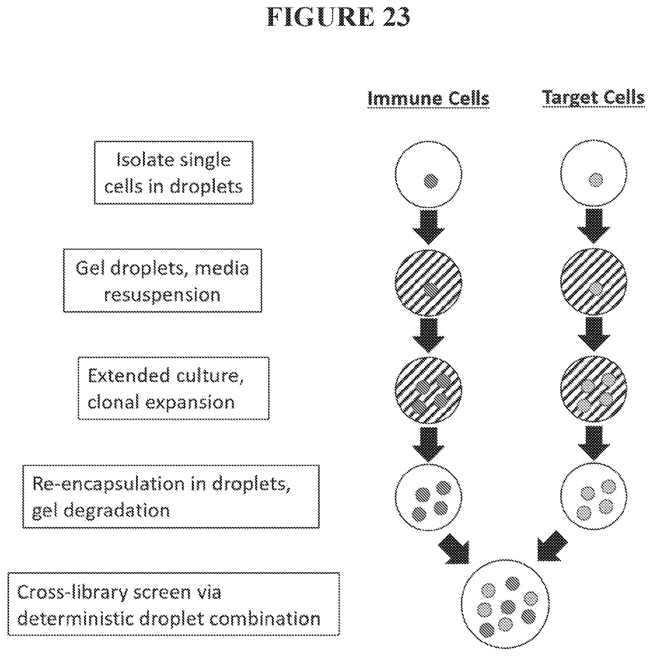

Jurkat cells were modified to express a library of T-cell receptor (TCRs) (Spindler et al, 2020, Nature Biotechnology, 38(5), 609-619). Similarly, COS-7 cells (African green monkey kidney fibroblast-like cell line) were co-transfected with HLA class I alleles and a library of cancer-neoantigen expressing tandem minigenes to serve as artificial antigen presenting cells (APCs) (Lu et al, 2014, Clinical Cancer Research, 20(13), 3401-3410). Each cell library was encapsulated in microfluidic droplets at 10% occupancy with 1% low melting point agarose (Fisher Scientific) using an 80 um co-flow dropmaker. After encapsulation, the droplets were cooled to 5 C to solidify the agarose, after which the emulsion is broken, and the resulting gel beads separated via centrifugation (FIG. 23). Subsequently, the gel beads were resuspended in media with appropriate growth factors and incubated. After 1 week of incubation at 37 C, the number of cells contained in each gel bead doubled several times and contained a clonal population of T-cells or APCs. Gel beads were then separated from bulk media via centrifugation and stained with CellTracker Blue (for Jurkats, Thermo Fisher) or CellTracker Green (for COS-7, Thermo Fisher). Beads are then re-encapsulated with in droplets with agarase (Thermo Fisher), which breaks down the gel and releases cells back into liquid phase. Next, combinatorial cell assays were created by deterministically combining droplets containing T-cells, APC, and IL-2 detection reagents. After 12 hours of incubation, droplets were detected, and sequencing reagents were deterministically merged with assay-positive droplets. Assays performed on small clonal populations of cells have greater sensitivity than single-cell assays because single-cell stochasticity is averaged among a greater number of cells and provide a larger amount of genomic material for sequencing.

### REFERENCES

[1] Bray, J. Ferlay, I. Soerjomataram, R. L. Siegel, L. A. Torre, and A. Jemal, “Global cancer statistics 2018: GLOBOCAN estimates of incidence and mortality worldwide for 36 cancers in 185 countries,” CA. Cancer J. Clin., vol. 68, no. 6, pp. 394-424, November 2018.
[2] Sudhakar, “History of Cancer, Ancient and Modern Treatment Methods,” J. Cancer Sci. Ther., vol. 01, no. 02, pp. i-iv, 2009.
[3] Tomasetti, Cristian, Li, Lu and Vogelstein, “Stem cell divisions, somatic mutations, cancer etiology, and Cancer Prevention,” Science (80-.), vol. 3556, no. March, pp. 1330-1334, 2017.
[4] Johnson et al., “Gene therapy with human and mouse T-cell receptors mediates cancer regression and targets normal tissues expressing cognate antigen,” Blood, vol. 114, no. 3, pp. 535-546, July 2009.
[5] Parkhurst et al., “T cells targeting carcinoembryonic antigen can mediate regression of metastatic colorectal cancer but induce severe transient colitis,” Mol. Ther., vol. 19, no. 3, pp. 620-626, March 2011.
[6] BrAunlein, Eva and Krackhardt, “Identification and Characterization of Neoantigens As Well As Respective Immune Responses in Cancer Patients,” Front. Immunol., vol. 8 (1702), 2017.
[7] Amarosi, “No Title,” Healio Heme Onc Today, 2019.
[8] Sahin et al., “Personalized RNA mutanome vaccines mobilize poly-specific therapeutic immunity against cancer,” Nature, vol. 547, no. 7662, pp. 222-226, July 2017.
[9] Liang et al., “The common neoantigens in colorectal cancer are predicted and validated to be presented or immunogenic,” bioRxiv, pp. 1-47, 2019.
[10] Bulik-Sullivan et al., “Deep learning using tumor HLA peptide mass spectrometry datasets improves neoantigen identification,” Nat. Biotechnol., vol. 37, no. 1, pp. 55-71, 2019.
[11] Lu et al., “Efficient identification of mutated cancer antigens recognized by T cells associated with durable tumor regressions,” Clin. Cancer Res., vol. 20, no. 13, pp. 3401-3410, July 2014.
[12] Bethune and A. V Joglekar, “Personalized T cell-mediated cancer immunotherapy: progress and challenges,” Current Opinion in Biotechnology, vol. 48. pp. 142-152, December-2017.
[13] Garris et al., “Successful Anti-PD-1 Cancer Immunotherapy Requires T Cell-Dendritic Cell Crosstalk Involving the Cytokines IFN-γ and IL-12,” Immunity, vol. 49, no. 6, pp. 1148-1161.e7, December 2018.
[14] Ahn, J. Agresti, H. Chong, M. Marquez, and D. A. Weitz, “Electrocoalescence of drops synchronized by size-dependent flow in microfluidic channels,” Appl. Phys. Lett., vol. 88, no. 26, p. 264105, June 2006.
[15] Cole et al., “Printed droplet microfluidics for on demand dispensing of picoliter droplets and cells,” Proc. Natl. Acad. Sci., July 2017.
[16] Siltanen et al., “An Oil-Free Picodrop Bioassay Platform for Synthetic Biology,” Sci. Rep., vol. 8, no. November 2017, pp. 1-7, December 2018.
[17] Kim, I. C. Clark, P. Shahi, and A. R. Abate, “Single-Cell RT-PCR in Microfluidic Droplets with Integrated Chemical Lysis,” Anal. Chem., vol. 90, no. 2, pp. 1273-1279, 2018.
[18] Klein et al., “Droplet Barcoding for Single-Cell Transcriptomics Applied to Embryonic Stem Cells,” Cell, vol. 161, no. 5, pp. 1187-1201, May 2015.
[19] Gee et al., “Antigen Identification for Orphan T Cell Receptors Expressed on Tumor-Infiltrating Lymphocytes,” Cell, vol. 172, no. 3, pp. 549-563.e16, 2018.
[20] “Influenza Virus Control Peptide Pool.” [Online]. Available: https://www.anaspec.com/products/product.asp?id=44261.
[21] Dutoit et al., “Multiepitope CD8+ T cell response to an NY-ESO-1 peptide vaccine results in imprecise tumor targeting,” J. Clin. Invest., vol. 110, no. 12, pp. 1813-1822, December 2002.
[22] “Now Available: Anti-NY-ESO-1 T Cells|Astarte Biologics.” [Online]. Available: https://astartebio.com/blog/ny-eso-1-specific-t-cell-lines-now-available-for-cancer-immunotherapyresearch/.
[23] Britten, R. G. Meyer, T. Kreer, I. Drexler, T. Wölfel, and W. Herr, “The use of HLA-A*0201 transfected K562 as standard antigen-presenting cells for CD8+T lymphocytes in IFN-γ ELISPOT assays,” J. Immunol. Methods, vol. 259, no. 1-2, pp. 95-110, January 2002.
[24] Malyguine, S. Strobl, K. Dunham, M. R Shurin, and T. J. Sayers, “ELISPOT Assay for Monitoring Cytotoxic T Lymphocytes (CTL) Activity in Cancer Vaccine Clinical Trials,” Cells, vol. 1, pp. 111-126, 2012.
[25] Clontech, Takara, and cellartis, “SMARTer T-cell receptor profiling in single cells,” Web Document Reprint. [Online]. Available: http://catalog.takara-bio.co.jp/PDFS/w_human_sctcr_tn.pdf.
[26] Adler et al., “Rare, high-affinity anti-pathogen antibodies from human repertoires, discovered using microfluidics and molecular genomics,” MAbs, vol. 9, no. 8, pp. 1282-1296, 2017.
[27] Howie et al., “High-throughput pairing of T cell receptor α and β sequences,” Sci. Transl. Med., vol. 7, no. 301, pp. 301ra131-301ra131, August 2015.
[28] Sciambi and A. R. A. R. Abate, “Accurate microfluidic sorting of droplets at 30 kHz,” Lab Chip, vol. 15, no. 1, pp. 47-51, October 2014.
[29] Chevalier et al., “High-throughput monitoring of human tumor-specific T-cell responses with large peptide pools,” Oncoimmunology, vol. 4, no. 10, pp. 1-7, 2015.
[30] Deniger et al., “T-cell responses to TP53 ‘Hotspot’ Mutations and unique neoantigens expressed by human ovarian cancers,” Clin. Cancer Res., vol. 24, no. 22, pp. 5562-5573, 2018.
[31] Ali et al., “Induction of neoantigen-reactive T cells from healthy donors,” Nat. Protoc., vol. 14, no. 6, pp. 1926-1943, 2019.

### ADDITIONAL REFERENCES

[1] G. Rodgers et al., “Glimmers in illuminating the druggable genome,” Nat. Rev. Drug Discov., vol. 17, p. 301, January 2018.
[2] S.-H. Xiao et al., “High Throughput Screening for Orphan and Liganded GPCRs,” Combinatorial Chemistry & High Throughput Screening, vol. 11, no. 3. pp. 195-215, 2008.
[3] A. Nieto Gutierrez and P. H. McDonald, “GPCRs: Emerging anti-cancer drug targets,” Cell. Signal., vol. 41, no. September, pp. 65-74, 2018.
[4] B. G. Yadav, “Trends in Antibody Generation Techniques—the Fully Synthetic Human Combinatorial Antibody Library (HuCAL®) powerful,” 2015.
[5] C. McMahon et al., “Yeast surface display platform for rapid discovery of conformationally selective nanobodies,” Nat. Struct. Mol. Biol., vol. 25, no. 3, pp. 289-296, 2018.
[6] D. Huang and E. V. Shusta, “A yeast platform for the production of single-chain antibody-green fluorescent protein fusions,” Appl. Environ. Microbiol., vol. 72, no. 12, pp. 7748-7759, 2006.
[7] R H. Cole et al., “Printed droplet microfluidics for on demand dispensing of picoliter droplets and cells,” Proc. Natl. Acad. Sci., July 2017.
[8] C. A. Siltanen et al., “An Oil-Free Picodrop Bioassay Platform for Synthetic Biology,” Sci. Rep., vol. 8, no. 1, pp. 1-7, 2018.
[9] E. Z. Z. Macosko et al., “Highly Parallel Genome-wide Expression Profiling of Individual Cells Using Nanoliter Droplets,” Cell, vol. 161, no. 5, pp. 1202-1214, 2015.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method comprising:
a) flowing a first or second discrete entity in a carrier fluid in a microfluidic device,
wherein said microfluidic device comprises:
i) an inlet channel,
ii) a sorting channel in fluid communication with said inlet channel,
iii) first and second outlet channels in fluid communication with said sorting channel, wherein said first outlet channel comprises a merger region,
iv) a sorting element positioned in proximity to the sorting channel, and
v) a trapping element positioned in proximity to said merger region, and
wherein said first discrete entity comprises at least one surface display cell (SD cell) and is free of other types of cells, wherein each SD cell comprises:
i) an outer surface displaying a polypeptide, wherein said polypeptide comprises at least one neoantigen, and
ii) a first detectable label; and
wherein said second discrete entity comprises at least one T-cell and is free of other types of cells, wherein said T-cell comprises: i) a T-cell receptor (TCR), ii) nucleic acid encoding said TCR, and iii) a second detectable label; and
wherein said flowing causes said first or second discrete entity to pass through said inlet channel to said sorting channel;
b) selectively sorting said first or second discrete entity in the sorting channel to said first outlet channel;
c) trapping said first or second discrete entity in said merger region via said trapping element; and
d) repeating steps a)-c) such that both said first and second discrete entities are trapped at said merger region and combine to form a first combined discrete entity, wherein said repeating steps a)-c) overlaps in time, or is after, when said steps a)-c) are performed.

2. The method of claim 1, further comprising: e) detecting directly or indirectly, in said first combined discrete entity, whether said TCR on said T-cell sufficiently binds said neoantigen on said SD cell to activate said T-Cell.

3. The method of claim 2, wherein said detecting is performed when said first combined discrete entity is at said merger region.

4. The method of claim 2, further comprising: releasing said first combined discrete entity from said merger region such that it flows into a downstream area.

5. The method of claim 4, wherein said detecting is performed when said first combined discrete entity is at said downstream area.

6. The method of claim 5, wherein said downstream area is a collection area or a receptacle external to said microfluidic device.

7. The method of claim 1, wherein said SD cell, prior to step a) has been pulsed with said neoantigen.

8. The method of claim 7, wherein the identity of said neoantigen is known prior to performing the method.

9. The method of claim 1, wherein said SD cell further comprises: iii) a nucleic acid sequence encoding said neoantigen.

10. The method of claim 9, wherein said nucleic acid sequence is from a library of nucleic acid sequences encoding different neoantigens.

11. The method of claim 1, wherein said SD cell comprises an antigen presenting cell (APC).

12. The method of claim 1, wherein said SD cell: comprises one or more nucleic acid sequences encoding an MHC sequence and said neoantigen.

13. The method of claim 1, wherein said TCR of said T-cell is a chimeric antigen receptor.

14. The method of claim 1, wherein said TCR of said T-cell is endogenous to said T-cell or a TRC synthesized as part of a library.

15. The method of claim 1, wherein said combined discrete entity further comprises detection reagents for detecting activation molecule release from said T-cell when it is activated, wherein said activation molecule is selected from: a cytokine, CD107a, and Granzyme B.

16. The method of claim 15, wherein said detection reagents comprise first and second anti-activation molecule antibodies and/or granzyme B cleavable substrates.

17. The method of claim 16, wherein said second antibody is detectably labeled and wherein said first antibodies are attached to a bead.

18. The method of claim 1, wherein steps a)-d) are performed: A) in 2 milliseconds (mS) or less; B) is about 1 mS; C) in about 0.5-1.0 mS, D) in about 10-100 mS; or E) about 100-1000 mS

19. The method of claim 1, further comprising repeating steps a)-d) at least 99 times such that a total of at least 100 combined discrete entities are formed.

20. The method of claim 1, further comprising repeating steps a)-d) at least 999 times such that a total of at least 1000 combined discrete entities are formed.

21. The method of claim 1, further comprising repeating steps a)-d) at least 9999 times such that a total of at least 10000 combined discrete entities are formed.

22. The method of claim 1, further comprising repeating steps a)-d) at least 99,999 times such that a total of at least 100,000 combined discrete entities are formed.

23. The method of claim 19 or 20, wherein said 100 or said 1000 discrete entities are formed: A) in 2 seconds or less; B) is about 1 second; C) in about 30-60 seconds.

24. The method of claim 21 or 22, wherein said 10,000 or said 100,000 discrete entities are formed: A) in 20 seconds or less; B) is about 10 seconds; C) in about 300-600 seconds.

25. The method of claim 1, further comprising: e) detecting directly or indirectly, in said first combined discrete entity, that said neoantigen binds said TCR thereby activating said T-Cell.

26. The method of claim 25, further comprising: merging a third discrete entity with said first combined entity to generate a first further combined entity, wherein said third discrete entity comprises a lysis buffer, and wherein said at least one SD cell and said at least one T-cell are lysed inside said first further combined entity.

27. The method of claim 26, wherein said nucleic acid encoding said TCR is at least partially sequenced and/or wherein a nucleic acid sequence encoding the neoantigen is present and is at least partially sequenced.

28. The method of claim 26, further comprising: merging a fourth discrete entity with said first further combined entity to generate a first additionally combined entity, wherein said fourth discrete entity comprises: barcoded oligonucleotides and a polymerase.

29. The method of claim 28, wherein said barcoded oligonucleotide comprises barcoded template switch oligonucleotides (BTSOs), and wherein said polymerase comprises reverse transcriptase.

30. The method of claim 29, wherein said BTSOs are linked to a solid support bead via a photocleavable linker.

31. The method of claim 28, wherein said first additional combined entity further comprises primers specific for the alpha and/or beta regions of the TCR.

32. The method of claim 28, wherein said first additional combined entity further comprises primers specific for a nucleic acid sequence encoding the neoantigen.

33. The method of claim 1, wherein said first and/or second detectable marker comprises a fluorescent protein.

34. The method of claim 1, wherein said at least one SD cell and/or said at least one T-cell are mammalian cells or human cells.

35. The method of claim 1, wherein, further comprising releasing said first combined discrete entity from the discrete entity merger region by deactivating, decreasing, or reversing said trapping element such that first combined discrete entity flows out of said first outlet channel.

36. The method of claim 1, wherein said first discrete entity is flowed through a first inlet channel and said second discrete entity is flowed through a second inlet channel.

37. The method of claim 1, wherein the sorting element comprises a first sorting electrode that exert an electromagnetic force sufficient to sort a discrete entity in the sorting channel to the first outlet channel.

38. The method of claim 37, wherein the electromagnetic force is a dielectrophoretic force.

39. The method of claim 37, wherein the electromagnetic force is an electrophoretic force.

40. The method of claim 37, the microfluidic device further comprises a second and/or third sorting electrode.

41. The method of claim 40, wherein the first and second sorting electrodes are configured such that the first and second sorting electrodes form a bipolar electrode pair and the first trapping electrode is positively charged.

42. The method of claim 40, wherein the first and second sorting electrodes are positioned on opposite sides of the sorting channel.

43. The method of claim 40, wherein at least one of the following applies:
i) the first sorting electrode is positioned closer to the sorting channel than the second sorting electrode,
ii) the second sorting electrode is positioned closer to the sorting channel than the first sorting electrode,
iii) the distance between an end of the first sorting electrode, the second sorting electrode, or both and an interior wall of the sorter channel is between approximately 1 μm and approximately 100 μm,
iv) the distance between the first sorting electrode and the second sorting electrode is approximately 25 μm to approximately 500 μm,
v) the first sorting electrode and the second sorting electrode are connected to an alternating current electrical source with a frequency of approximately 0.1 kHz to approximately 100 kHz and a voltage of approximately 10 V to approximately 10,000 V,
vi) each sorting electrode comprises a liquid electrode,
vii) each sorting liquid electrode comprise one or more liquid channels imbedded in the method and filled with conductive media, and/or
viii) the sorting element comprises a valve, a surface wave sorting element, an acoustic streaming element, or a combination thereof.

44. The method of claim 1, wherein the trapping element exerts an electromagnetic force, exerts a mechanical force, or a combination thereof sufficient to trap said first and second discrete entities in the discrete entity merger region for a time sufficient for the discrete entities to combine to form a combined discrete entity.

45. The method of claim 1, wherein the trapping element comprises a first trapping electrode that exerts an electromagnetic force sufficient to trap discrete entities in the merger region for a time sufficient for discrete entities to combine to form a combined discrete entity.

46. The method of claim 44, wherein the electromagnetic force is a dielectrophoretic force.

47. The method of claim 46, wherein the electromagnetic force is an electrophoretic force.

48. The method of claim 1, wherein the microfluid device further comprises a second and/or third trapping electrode.

49. The method of claim 48, wherein the first and second trapping electrodes are configured such that the first and second trapping electrodes form a bipolar electrode pair and the first trapping electrode is positively charged.

50. The method of claim 48, wherein at least one of the following apply:
i) the first and second sorting electrodes are positioned on the same side of the sorting channel,
ii) the first trapping electrode is positioned closer to the first outlet channel than the second trapping electrode, or the second trapping electrode is positioned closer to the first outlet channel than the first trapping electrode,
iii) the distance between an end of the first trapping electrode, the second trapping electrode, or both and an interior wall the first outlet channel is between approximately 10 μm and approximately 50 μm,
iv) the distance between the first trapping electrode and the second trapping electrode is approximately 25 μm to approximately 500 μm,
v) the distance is approximately 50 μm to approximately 200 μm,
vi) the first trapping electrode and the second trapping electrode are connected to an alternating current electrical source with a frequency of approximately 0.1 kHz to approximately 100 kHz and a voltage of approximately 10 V to approximately 10,000 V,
vii) the frequency is approximately 1 kHz to approximately 50 kHz,
viii) each trapping electrode comprises a liquid electrode,
ix) each trapping liquid electrode comprise one or more liquid channels imbedded in the method and filled with conductive media,
x) the first trapping electrode electrodes extends along the first outlet channel downstream of the discrete entity merger region, and/or
xi) the second trapping electrode extends along the first outlet channel downstream of the discrete entity merger region.

51. The method of claim 1, wherein the sorting channel defines a concentric or approximately concentric flow path, and wherein a portion of the first sorting electrode is located at the center of the concentric or approximately concentric flow path.

52. The method of claim 1, wherein the sorting element is positioned closer to the first outlet channel than to the second outlet channel.

53. The method of claim 1, wherein the microfluidic device further comprises a partial height flow divider positioned in the sorting channel, wherein the partial height flow divider is configured to direct a discrete entity towards the first outlet channel or the second outlet channel.

54. The method of claim 53, wherein the height of the partial height flow divider is approximately 50% to 75% of the height of the sorting channel.

55. The method of claim 1, wherein the discrete entity merger region comprises a feature selected from the group consisting of: a geometric change in a dimension of the first outlet channel, a flow obstacle, a flow divider, a laminating fluid inlet, a valve, or a combination thereof.

56. The method of claim 1, wherein the discrete entity merger region comprises a geometric change in a dimension of the first outlet channel, and wherein the geometric change comprises an increase in the cross-sectional area of the first outlet channel.

57. The method of claim 1, where the discrete entity merger region comprises a geometric change, and wherein the geometric change comprises a recess in a wall of the first outlet channel.

58. The method of claim 1, wherein the discrete entity merger region comprises a laminating fluid inlet channel configured such that flowing laminating fluid through the laminating fluid inlet channel will direct a discrete entity in the discrete entity merger region towards a trapping electrode.

59. The method of claim 1, wherein the inlet channel comprises an upstream region located between the sorting channel and the discrete entity merger region, and wherein the change in cross-sectional area is such that the discrete entity merger region has a larger cross-sectional area than the upstream region.

60. The method of claim 1, wherein the discrete entity merger region has a triangular shape, an approximately triangular shape, a trapezoidal shape, or an approximately trapezoidal shape defined by channel walls.

61. The method of claim 1, wherein the discrete entity merger region comprises a valve, wherein the valve is a membrane valve configured to impede the flow of a discrete entity past the discrete entity merger region while allowing flow of the carrier fluid past the discrete entity merger region in a first state, and wherein the membrane valve is configured to release the discrete entity or a combined discrete entity in a second state.

62. The method of claim 1, wherein the microfluidic device further comprises a spacer fluid channel in fluid communication with the inlet channel, wherein the spacer fluid channel is configured such that flowing spacer fluid through the spacer fluid channel causes spacer fluid to be located between said first and second discrete entities flowing through the inlet channel, thereby maintaining or increasing the distance between the first and second discrete entities, and thereby allowing each of first and second discrete entities to be independently sorted or not sorted.

63. The method of claim 1, wherein the first and second discrete entities are droplets.

64. The method of claim 63, wherein said droplets comprise an aqueous fluid which is immiscible in said carrier fluid.

65. The method of claim 1, wherein said carrier fluid comprises oil.

66. The method of claim 63, wherein the carrier fluid is an aqueous fluid and the droplets comprise a fluid which is immiscible with the carrier fluid.

67. The method of claim 1, wherein said discrete entities have a dimension of from about 1 μm to about 1000 μm.

68. The method of claim 1, wherein the discrete entities have a diameter of from about 1 μm to 1000 μm.

69. The method of claim 1, wherein the discrete entities have a volume of from about 1 femtoliter to about 1000 nanoliters, or from 10 to 800 picoliters.

70. A composition, system, or kit comprising:
a) first and second discrete entities in a carrier fluid, and/or
b) a combined discrete entity in a carrier fluid, wherein said combined discrete entity is a combination of said first and second discrete entities,
wherein said first discrete entity comprises at least one surface display cell (SD cell) and is free of other types of cells, wherein each SD cell comprises:
i) an outer surface displaying a polypeptide, wherein said polypeptide comprises at least one neoantigen, and
ii) a first detectable label; and
wherein said second discrete entity comprises at least one T-cell and is free of other types of cells, wherein said T-cell comprises: i) a T-cell receptor (TCR), ii) nucleic acid encoding said TCR, and iii) a second detectable label.

71. The composition, system or kit of claim 70, further comprising: c) a microfluidic device comprising:
i) an inlet channel,
ii) a sorting channel in fluid communication with said inlet channel,
iii) first and second outlet channels in fluid communication with said sorting channel, wherein said first outlet channel comprises a merger region,
iv) a sorting element positioned in proximity to the sorting channel, and
v) a trapping element positioned in proximity to said merger region.

72. The composition, system, or kit of claim 70, further comprising a plurality of said first discrete entities and/or a plurality of said second discrete entities.

73. The composition, system, or kit of claim 70, wherein each of said first and second discrete entities comprises a droplet, and/or wherein said combined discrete entity comprises a droplet.

74. The composition, system, or kit of claim 73, wherein said droplet comprise an aqueous fluid in said carrier fluid.

75. The composition, system, or kit of claim 74, wherein said carrier fluid comprises oil.

76. The composition, system, or kit of claim 70, wherein at least one of said first and second discrete entities, or said combined discrete entity, further comprises at least one of the following: a bead, primer, barcode sequence, template switching oligonucleotide (TSO), or a reverse transcriptase.

77. The composition, system, or kit of claim 70, wherein said SD cell has been pulsed with said neoantigen.

78. The composition, system, or kit of claim 70, wherein said SD cell further comprises: iii) a nucleic acid sequence encoding said neoantigen.

79. The composition, system, or kit of claim 70, wherein said SD cell comprises an antigen presenting cell (APC).

80. The composition, system, or kit of claim 70, wherein said SD cell: comprises one or more nucleic acid sequences encoding an MHC sequence and said neoantigen.

81. The composition, system, or kit of claim 70, wherein said TCR of said T-cell is a chimeric antigen receptor.

82. The composition, system, or kit of claim 70, wherein said TCR of said T-cell is endogenous to said T-cell.

83. The composition, system, or kit of claim 70, wherein at least one of said first and second discrete entities, or said combined discrete entity, further comprises detection reagents for detecting activation molecule release from said T-cell when it is activated.

84. The composition, system, or kit of claim 83, wherein said detection reagents comprise first and second anti-activation molecule antibodies and/or granzyme B cleavable substrates.

85. The composition, system, or kit of claim 84, wherein said second antibody is detectably labeled and wherein said first antibodies are attached to a bead.

86. The composition, system, or kit of claim 70, wherein one and only one T-cell is present in first or second discrete entity or the combined discrete entity.

87. The composition, system, or kit of claim 70, wherein one and only one SD cell is present in first or second discrete entity or the combined discrete entity.

88. A method comprising:
a) flowing a plurality of discrete entities in a carrier fluid in a microfluidic device,
wherein said microfluidic device comprises: i) a sorting channel, ii) first and second outlet channels in fluid communication with said sorting channel, iii) a collection area in fluid communication with said first outlet channel, and iv) a discard area in fluid communication with said second outlet channel,
wherein each of said plurality of discrete entities comprises: i) an activated cell which is an activated T-cell or activated Natural Killer (NK) cell), and ii) detection reagents for detecting interferon-gamma (IFN-γ) release and/or interleukin-6 (IL-6) release from said activated T-cell;
b) detecting directly or indirectly, in each of said plurality of discrete entities when present in said sorting channel whether said activated cell: i) releases IFN-γ (IFN-γ positive discrete entity), or ii) does not release IL-6 (IL-6 negative discrete entity), or iii) releases IFN-γ and does not release IL-6 (IFN-γ positive and IL-6 negative discrete entity),
c) selectively sorting each of said plurality of discrete entities in the sorting channel:
i) to said first outlet channel and into said collection area if said discrete entity is an IFN-γ positive discrete entity, and to said second outlet channel and into said discard area if said discrete entity is not an IFN-γ positive discrete entity, thereby generating a population of IFN-γ positive discrete entities in said collection area; and/or
ii) to said first channel and into said collection area if said discrete entity is an IL-6 negative discrete entity, and to said second channel and into said discard area if said discrete entity is not an TL-6 negative discrete entity, thereby generating a population of IL-6 negative discrete entities in said collection area; and/or
iii) to said first channel and into said collection area if said discrete entity is an IFN-γ positive and IL-6 negative discrete entity, and to said second channel and into said discard area if said discrete entity is not IFN-γ positive and TL-6 negative, thereby generating a population of IFN-γ positive and IL-6 negative discrete entities in said collection area; and
d) treating at least a portion of: i) said population of IFN-γ positive discrete entities, and/or ii) said population of IL-6 negative discrete entities, and/or iii) said population of IFN-γ positive and IL-6 negative discrete entities; under conditions such that some or all of said activated cells therein are each transduced with a vector encoding a chimeric antigen receptor thereby generating: i) a population of IFN-γ positive CAR T-cells or CAR NK cells, and/or ii) a population of IL-6 negative CAR T-cells or CAR NK cells, and/or iii) a population of IFN-γ positive, IL-6 negative, CAR T-cells or CAR NK cells.

89. The method of claim 88, wherein each of said plurality of discrete entities contains only one of said activated cells.

90. The method of claim 88, wherein each of said plurality of discrete entities is in the form of a droplet.

91. The method of claim 90, wherein said droplet comprises an emulsion.

92. The method of claim 91, wherein said treating comprises breaking said emulsion of each of plurality of discrete entities prior to said activated cells being transduced by said vector.

93. The method of claim 88, wherein each of said discrete entities comprises at least one activation molecule selected from the group consisting of: i) an anti-CD3 antibody, ii) an active fragment of said anti-CD3 antibody, iii) an anti-CD28 antibody, and iv) an active fragment of said anti-CD28 antibody.

94. The method of claim 88, further comprising after c), but before d) flowing a fraction of: i) said population of IFN-γ positive discrete entities, and/or ii) said population of IL-6 negative discrete entities, and/or said IFN-γ positive and IL-6 negative discrete entities; in a carrier fluid in a microfluidic device such that reagent-containing discrete entities merge with said IFN-γ positive discrete entities and/or said IL-6 negative discrete entities and/or said IFN-t positive and IL-6 negative discrete entities to generate a population of combined entities, wherein said reagent-containing discrete entities comprises lysis buffer and sequencing reagents.

95. The method of claim 94, wherein said sequencing reagents comprise barcoded oligonucleotides and a polymerase.

96. The method of claim 95, wherein said barcoded oligonucleotide comprises barcoded template switch oligonucleotides (BTSOs), and wherein said polymerase comprises reverse transcriptase.

97. The method of claim 96, wherein said BTSOs are linked to a solid support bead via a photocleavable linker.

98. The method of claim 94, further comprising, prior to step d) performing expression analysis sequencing on at least some of said activated cells from said population of combined entities.

99. The method of claim 98, wherein said expression analysis generates data regarding at least cytokine gene toxic to a patient if over expressed by a CAR T-cell or CAR NK cell when injected into a subject.

100. The method of claim 99, wherein said at least one cytokine is LGALS1.

101. The method of claim 98, wherein said expression analysis generates data regarding an inflammatory signature profile that indicates said CAR T-cell or CAR NK cell is toxic to a patient if injected into a subject.

102. The method of claim 98, wherein said expression analysis generates data regarding expression levels for at least two beneficial cytokines, wherein over-expression of said at least two cytokines identifies said activated cell as polyfunctional.

103. The method of claim 98, wherein said expression analysis generates data regarding a polyfunctional signature profile.

104. The method of claim 103, wherein said polyfunctional signature profile comprises at least two genes selected from the group consisting of: ANXA1, CCL1, CCL3, CCL4, CCL5, CD40LG, CSF2, GZMA, GZMB, ICOS, IFNG, IL2, IL2RA, IL13, IL32, LCK, TNFRSF9, TNFRSF18, TNFRSF4, and TNFRSF14.

105. The method of claim 98, wherein said expression analysis generates data regarding T-cell identity for at least one, or all, of the following genes: CCND2, CD2, CD28, CD247, CD3D, CD3E, CD3G, CD44, CD7, CD96, TRAC, TRAV and TRBV.

106. A method comprising:
a) flowing a plurality of discrete entities in a carrier fluid in a microfluidic device,
wherein said microfluidic device comprises: i) a sorting channel, ii) first and second outlet channels in fluid communication with said sorting channel, iii) a collection area in fluid communication with said first outlet channel, and iv) a discard area in fluid communication with said second outlet channel,
wherein each of said plurality of discrete entities comprises: i) an activated cell which is an activated T-cell or activated Natural Killer cell), and ii) detection reagents for detecting release of at least two types of cytokines from said activated cell;
b) detecting directly or indirectly, in each of said plurality of discrete entities when present in said sorting channel whether said activated cell releases said at least two types of cytokines (polyfunctional discrete entities) or does not release said at least two types of cytokines (non-polyfunctional discrete entities); and
c) selectively sorting each of said plurality of discrete entities in the sorting channel:
i) to said first outlet channel and into said collection area if said discrete entity is a polyfunctional discrete entity, and
ii) to said second outlet channel and into said discard area if said discrete entity is a non-polyfunctional discrete entity,
thereby generating a population of polyfunctional discrete entities in said collection area.

107. The method of claim 106, wherein said activated cells are CAR T-cells.

108. The method of claim 106, wherein said activated cells are TCR T-cells.

109. A method comprising:
a) flowing a plurality of aqueous discrete entities in an oil carrier fluid in an emulsion inlet channel of a microfluidic device,
wherein said inlet channel feeds into an emulsion-aqueous junction, wherein: i) an aqueous inlet channel feeds into said emulsion-aqueous junction; ii) an aqueous outlet channel branches off of said emulsion-aqueous junction; and iii) an emulsion outlet channel branches off of said emulsion-aqueous junction,
wherein an aqueous carrier fluid flows from said aqueous inlet channel to said emulsion-aqueous junction to said aqueous outlet channel and a first electrical signal is applied to said aqueous carrier fluid,
wherein each of said plurality of aqueous discrete entities in said oil carrier merge into said aqueous carrier fluid at said emulsion-aqueous junction and flow out said aqueous outlet channel when said first electrical signal is applied to said aqueous carrier fluid,
b) detecting at least one of said discrete entities as desired (desired discrete entity) by detecting at least one agent present in said desired discrete entity prior to it reaching said emulsion-aqueous junction, and
c) changing said first electrical signal to a second electrical signal applied to said aqueous carrier fluid such that said desired discrete entity is prevented from merging into said aqueous carrier fluid at said emulsion-aqueous junction, and instead, flows in said carrier oil into said emulsion outlet.

110. The method of claim 109, further comprising: d) processing said desired discrete entity as described herein.


SEQUENCE LISTING (9 sequences)

SEQ ID NO: 1 - DNA, 25 bp, Artificial Sequence
  Synthetic
cagacagacttgtcactggatttag

SEQ ID NO: 2 - DNA, 23 bp, Artificial Sequence
  Synthetic
cttttgggtgtgggagatctctg

SEQ ID NO: 3 - DNA, 61 bp, Artificial Sequence
  Synthetic
  misc_feature (36)..(37): n is a, c, g, or t
tgtctcgtgggctcggagatgtgtataagagacagnngtcactggatttagagtctctcag

SEQ ID NO: 4 - DNA, 62 bp, Artificial Sequence
  Synthetic
  misc_feature (1)..(2): n is a, c, g, or t
  misc_feature (38)..(39): n is a, c, g, or t
nntgtctcgtgggctcggagatgtgtataagagacagnngagatctctgcttctgatggctc

SEQ ID NO: 5 - DNA, 56 bp, Artificial Sequence
  Synthetic
  misc_feature (34)..(35): n is a, c, g, or t
tcgtcggcagcgtcagatgtgtataagagacagnntatcaacgcagagtgaatggg

SEQ ID NO: 6 - DNA, 21 bp, Artificial Sequence
  Synthetic
tatcaacgcagagtgaatggg

SEQ ID NO: 7 - DNA, 30 bp, Artificial Sequence
  Synthetic
aagcagtggtatcaacgcagagtgaatggg

SEQ ID NO: 8 - DNA, 23 bp, Artificial Sequence
  Synthetic
aagcagtggtatcaacgcagagt

SEQ ID NO: 9 - DNA, 65 bp, Artificial Sequence
  Synthetic
  misc_feature (26)..(33): n is a, c, g, or t
  misc_feature (65)..(65): n is a, c, g, or t
aagcagtggtatcaacgcagagtacnnnnnnnnttttttttttttttttttttttttttttttvn